SEQUENCE LISTING (4 sequences)

SEQ ID NO: 1 - protein, 15 aa, synthetic construct
LQWRRNFGVWARYRL

SEQ ID NO: 2 - protein, 20 aa, synthetic construct
LQWRRDDNVHNFGVWARYRL

SEQ ID NO: 3 - protein, 5 aa, synthetic construct
GGGGS

SEQ ID NO: 4 - protein, 5 aa, synthetic construct
SGGGG

(19) United States
(12) Patent Application Publication
(10) Pub. No.: US 2026/0295060 A1
(43) Pub. Date: Oct. 1, 2026

(54) NEU5GC-LEWIS A-TARGETING MOLECULES AND USES THEREOF IN CANCER

(71) Applicant: SRI International, Menlo Park, CA (US)

(72) Inventors: Indu VENUGOPAL, Harrisonburg, VA (US); Shelby KNOCHE, Rockingham, VA (US); Michael J. McGUIRE, Harrisonburg, VA (US); Kathlynn Corinne BROWN, Staunton, CA (US)

(73) Assignee: SRI International, Menlo Park, CA (US)

(21) Appl. No.: 19/489,405

(22) PCT Filed: Jun. 25, 2024
(86) PCT No.: PCT/US2024/035392

§ 371 Date: Dec. 2, 2025

Related U.S. Application Data
(60) Provisional application No. 63/510,326, filed on Jun. 26, 2023.

(51) Int. Cl. A61K 47/64; C07K 7/08; G01N 33/575

(52) U.S. Cl. CPC A61K 47/64; C07K 7/08; G01N 33/5759

(57) ABSTRACT

The disclosure relates to the discovery of Neu5Gc-Lewis^{a} as a biomarker for cancer cells and tissues and provides methods for diagnosis, treatment, and drug targeting based on the same, as well as molecules that specifically bind to Neu5Gc-Lewis^{a}.

### CROSS REFERENCE TO RELATED APPLICATIONS

This application claims priority to U.S. Provisional Application No. 63/510,326, filed Jun. 26, 2023, which is hereby incorporated by reference herein in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The Sequence Listing is written in the accompanying XML file titled: “206541-0005-00WO Sequence Listing.xml”; created on Jun. 21, 2024, and 4,608 bytes in size.

### FIELD

The disclosure relates to the discovery of Neu5Gc-Lewis^{a} as a biomarker for cancer cells, exosomes and tissues and provides methods for diagnosis, treatment, and drug targeting based on the same, as well as molecules that specifically bind to Neu5Gc-Lewis^{a}.

### BACKGROUND

Biomarkers play a crucial role in cancer research, diagnosis, prognosis, and treatment. They may help identify the presence of cancer at an early stage, even before symptoms become apparent. Early detection allows for timely intervention and potentially better treatment outcomes. Biomarkers may also provide valuable information about the prognosis or likely outcome of a particular cancer. They may help determine the aggressiveness of the disease, the risk of recurrence, and the overall survival rate. Moreover, biomarkers allow for targeted drug delivery, which significantly improves efficacy of cancer therapy. Drug targeting allows for the delivery of therapeutic agents directly to cancer cells or tumor sites, minimizing damage to healthy tissues. By targeting specific molecular markers or receptors expressed on cancer cells, therapeutics may selectively bind and exert their effects on the tumor, reducing off-target effects and associated side effects. Targeted therapies may be combined with other targeted agents or conventional chemotherapy drugs to create synergistic effects. By targeting multiple pathways or mechanisms simultaneously, combination therapies may enhance treatment efficacy and overcome limitations associated with single-agent therapies. Current targeting agents are primarily either small-molecule drugs or monoclonal antibodies. Small molecule drugs may enter cancer cells easily, but they cannot target cancer cells specifically. On the other hand, some monoclonal antibodies have the ability to target specific proteins found on the surface of cancer cells, but they generally do not have the ability to internalize into them. There is a need for molecules that may serve as disease biomarkers and do both—target as well as internalize into cancer cells.

### SUMMARY

Biomarkers and targeted drug delivery significantly improves efficacy of cancer therapy. In one embodiment, the disclosure provides that Neu5Gc-α-2,3-Gal-β-1,3-(Fuc-α-1,4)-GlcNAc-β, or Neu5Gc-Lewis^{a}, is a biomarker for cancer cells and tissues. Accordingly, the disclosure provides that Neu5Gc-Lewis^{a} may be used as a biomarker for early cancer detection and drug targeting. Furthermore, the disclosure also provides agents that may specifically target cancer cells by specifically binding Neu5Gc-Lewis^{a}, such as peptides and antibodies. In one embodiment, these peptides comprise MGS5, which may be MGS5_V1 and MGS5_V2. In one embodiment, the disclosure provides that both the MGS5_V1 and MGS5_V2 peptides may be conjugated to a therapeutic agent (such as an anti-cancer drug), allowing it to specifically target the tumor cells in a patient. This has several advantages including improved therapeutic efficacy while avoiding systemic toxicity.

Reference will now be made in detail to embodiments of the present application, one or more examples of which are described below. Each example is provided by way of explanation, not limitation of the application. In fact, it will be apparent to those skilled in the art that various modifications and variations may be made in the present application without departing from the scope or spirit of the application. For example, features illustrated or described as part of one embodiment may be used on another embodiment to yield a still further embodiment.

Thus, it is intended that the present application cover such modifications and changes as come within the scope of the appended claims and their equivalents. Other objects, features and embodiments of the present application are disclosed in or are apparent from the following detailed description. It is to be understood by those of ordinary skill in the art that the present discussion is a description of exemplary embodiments only and is not intended to limit the broader embodiments of the application.

Embodiment 1: A method of detecting Neu5Gc-Lewis^{a} on the surface and/or extracellular matrix of a cell or tissue, comprising (a) obtaining or having obtained a cell or tissue sample; (b) detecting whether the Neu5Gc-Lewis^{a} is present on the surface and/or extracellular matrix of the cell or tissue by contacting the cell or tissue sample with a molecule known to bind Neu5Gc-Lewis^{a} and that does not bind to any other form of Neu5Gc or any other Sialyl-Lewis^{a}, including free Neu5Gc, also referred to henceforth as a Neu5Gc-Lewis^{a}-binding molecule, and detecting binding between Neu5Gc-Lewis^{a} and the Neu5Gc-Lewis^{a}-binding molecule on the surface and/or extracellular matrix of the cell or tissue, optionally wherein the cell or tissue sample is obtained from a subject; and, optionally further comprising administering an anti-cancer therapy to the subject.

Embodiment 2: The method of embodiment 1, wherein the Neu5Gc-Lewis^{a}-binding molecule comprises a peptide, antibody, nucleic acid, a carbohydrate, a lipid, a small organic molecule, a lipid particle (e.g., nanoparticle), or an aptamer.

Embodiment 3: The method of embodiment 1 or 2, wherein the peptide has at least 75%, 80%, 85%, 90%, 95%, or 100% homology or identity to a peptide comprising or consisting of the sequence of SEQ ID NO:1 or SEQ ID NO:2.

Embodiment 4: The method of embodiment 1, 2, or 3, wherein the Neu5Gc-Lewis^{a}-binding molecule does not bind any other form of Neu5Gc or any other Sialyl-Lewis^{a}, including free Neu5Gc.

Embodiment 5: The method of any one of embodiments 1 to 4, wherein the binding takes place in vitro.

Embodiment 6: The method of any one of embodiments 1 to 5, wherein the cell is a cancer cell.

Embodiment 7: A method of identifying a cell, exosome or tissue as a cancer cell, exosome or tissue or diagnosing cancer in a subject, comprising detecting the presence of Neu5Gc-Lewis^{a} in a cell, exosome or tissue sample from the subject, wherein the presence of Neu5Gc-Lewis^{a} in the cell, exosome or tissue indicates the presence of a cancer cell, exosome or tissue and diagnosis of cancer in the subject, optionally further comprising administering an anti-cancer therapy to the subject.

Embodiment 8: The method of embodiment 7, wherein the presence of Neu5Gc-Lewis^{a} in the cell, exosome or tissue is detected with a Neu5Gc-Lewis^{a}-binding molecule that does not bind to any other form of Neu5Gc or any other Sialyl-Lewis^{a}, including free Neu5Gc.

Embodiment 9: The method of embodiment 8, wherein the Neu5Gc-Lewis^{a}-binding molecule comprises a peptide, antibody, nucleic acid, a carbohydrate, a lipid, a small organic molecule, a lipid particle (e.g., nanoparticle), or an aptamer.

Embodiment 10: The method of embodiment 9, wherein the peptide has at least 75%, 80%, 85%, 90%, 95%, or 100% homology or identity to a peptide comprising or consisting of the sequence of SEQ ID NO:1 or SEQ ID NO:2.

Embodiment 11: The method of any one of embodiments 7 to 10, wherein the cancer is pancreatic cancer, breast cancer, colon cancer, brain cancer, endometrial cancer, liver cancer, lung cancer, ovarian cancer, prostate cancer, stomach cancer, or urinary bladder cancer.

Embodiment 12: The method of embodiment 11, wherein the presence of Neu5Gc-Lewis^{a} discriminates between normal and cancerous tissue.

Embodiment 13: A method of targeted delivery of a cargo molecule to a cell and/or tissue, comprising contacting the cell or tissue with a Neu5Gc-Lewis^{a}-binding molecule linked to a cargo, directly or through a linker, wherein the Neu5Gc-Lewis^{a}-binding molecule (preferably, a peptide, antibody, nucleic acid, a carbohydrate, a lipid, a small organic molecule, a lipid particle (e.g., nanoparticle), or an aptamer) has been previously determined not to bind to any other form of Neu5Gc or any other Sialyl-Lewis^{a}, including free Neu5Gc, other than Neu5Gc-Lewis^{a}, preferably wherein the cell or tissue is a cancer cell or tissue and/or preferably wherein the cargo is an anti-cancer therapy/agent.

Embodiment 14: The method of embodiment 13, wherein the Neu5Gc-Lewis^{a}-binding molecule comprises a peptide having at least 75%, 80%, 85%, 90%, 95%, or 100% homology or identity to a peptide comprising or consisting of the sequence of SEQ ID NO:1 or SEQ ID NO:2 to detect Neu5Gc-Lewis^{a} on the cell or tissue.

Embodiment 15: The method of embodiment 13 or 14, wherein the cancer is pancreatic cancer, breast cancer, colon cancer, brain cancer, endometrial cancer, liver cancer, lung cancer, ovarian cancer, prostate cancer, stomach cancer, or urinary bladder cancer; preferably, wherein the targeted delivery of the cargo to said cancer is therapeutic against said cancer.

Embodiment 16: The method of any one of embodiments 13 to 15, wherein the cargo comprises a molecule selected from peptides, nucleic acids (e.g., siRNA, antisense oligonucleotides, microRNA, or shRNA), proteins (including, but not limited to, streptavidin, phycoerythrin, a cytokine, an interleukin, an enzyme, a receptor, a microprotein, a hormone, erythropoietin, a ribonuclease (RNAse), a deoxyribonuclease (DNAse)), a blood clotting factor, an anticoagulant, a bone morphogenetic protein, an engineered protein scaffold, a thrombolytic protein, a CRISPR protein, granulocyte-macrophage colony-stimulating factor (GMCSF), a transcription factor, a transposon, a reverse transcriptase, a viral interferon antagonist), fluorophores, liposomes, polysaccharides, nanoparticles, small molecule therapeutics (including, but not limited to, MMAF, DM1, Amanitin, Duocarmycin, doxorubicin, paclitaxel), PET dyes, NIR dyes, MRI agents, antibodies, ribonucleoproteins, antigenic peptides, protein toxins (including, but not limited to, saporin), a genome editing system.

Embodiment 17: A method of detecting extracellular or cell-surface or tissue-bound Neu5Gc-Lewis^{a} in a subject, comprising (a) obtaining or having obtained a sample of solid tissue, blood, serum, plasma, lavage, urine, milk, CSF, etc. from the subject; and (b) detecting whether Neu5Gc-Lewis^{a} is present in the sample by contacting the sample with a Neu5Gc-Lewis^{a}-binding molecule, preferably a peptide, antibody, nucleic acid, a carbohydrate, a lipid, a small organic molecule, a lipid particle (e.g., nanoparticle), or an aptamer, wherein the molecule does not bind any other form of Neu5Gc or any other Sialyl-Lewis^{a}, including free Neu5Gc, and detecting binding between the Neu5Gc-Lewis^{a}-binding molecule and the Neu5Gc-Lewis^{a} in the sample; optionally, further comprising administering an anti-cancer therapy to the subject.

Embodiment 18: The method of embodiment 17, wherein the presence of extracellular, cell-surface or tissue-bound Neu5Gc-Lewis^{a} indicates the presence of cancer in the subject.

Embodiment 19: The method of embodiment 17 or 18, wherein the Neu5Gc-Lewis^{a}-binding molecule comprises a peptide having at least 75%, 80%, 85%, 90%, 95%, or 100% homology or identity to a peptide comprising or consisting of the sequence of SEQ ID NO:1 or SEQ ID NO:2.

Embodiment 20: The method of any one of embodiments 17 to 19, wherein detection of extracellular or cell-surface or tissue-bound Neu5Gc-Lewis^{a} may discriminate cancer patients from cancer-free individuals and/or monitor cancer progression in the subject.

Embodiment 21: The method of any one of embodiments 17 to 20, wherein the cancer is pancreatic cancer, breast cancer, colon cancer, brain cancer, endometrial cancer, liver cancer, lung cancer, ovarian cancer, prostate cancer, stomach cancer, or urinary bladder cancer.

Embodiment 22: A method of detecting whether a disease is associated with the presence of Neu5Gc-Lewis^{a}, comprising (a) obtaining or having obtained diseased tissue from a subject and (b) contacting the diseased tissue with a Neu5Gc-Lewis^{a}-binding molecule and detecting the binding between the Neu5Gc-Lewis^{a}-binding molecule, preferably a peptide, antibody, nucleic acid, a carbohydrate, a lipid, a small organic molecule, a lipid particle (e.g., nanoparticle), or an aptamer, and the Neu5Gc-Lewis^{a} in the diseased tissue, wherein the molecule does not bind to any other form of Neu5Gc or any other Sialyl-Lewis^{a}, including free Neu5Gc.

Embodiment 23: A method of detecting atherosclerosis in a tissue, comprising contacting the tissue with a Neu5Gc-Lewis^{a}-binding molecule, preferably a peptide, antibody, nucleic acid, a carbohydrate, a lipid, a small organic molecule, a lipid particle (e.g., nanoparticle), or an aptamer, and detecting the binding between the Neu5Gc-Lewis^{a}-binding molecule and the Neu5Gc-Lewis^{a} in the vessel wall in the tissue, thereby detecting atherosclerosis in the vessel wall, wherein the molecule does not bind any other form of Neu5Gc or any other Sialyl-Lewis^{a}, including free Neu5Gc.

Embodiment 24: The method of embodiment 22 or 23, wherein the Neu5Gc-Lewis^{a}-binding molecule comprises a peptide having at least 75%, 80%, 85%, 90%, 95%, or 100% homology or identity to a peptide comprising or consisting of the sequence of SEQ ID NO:1 or SEQ ID NO:2.

Embodiment 25: A composition comprising a Neu5Gc-Lewis^{a}-binding molecule, preferably a peptide, antibody, nucleic acid, a carbohydrate, a lipid, a small organic molecule, a lipid particle (e.g., nanoparticle), or an aptamer, and a carrier or diluent, wherein the Neu5Gc-Lewis^{a}-binding molecule does not bind to any other form of Neu5Gc or any other Sialyl-Lewis^{a}, including free Neu5Gc.

Embodiment 26: The composition of embodiment 25, wherein the Neu5Gc-Lewis^{a}-binding molecule comprises a peptide having at least 75%, 80%, 85%, 90%, 95%, or 100% homology or identity to a peptide comprising or consisting of the sequence of SEQ ID NO:1 or SEQ ID NO:2.

Embodiment 27: The composition of embodiment 25 or 26, wherein the Neu5Gc-Lewis^{a}-binding molecule is chemically modified.

Embodiment 28: The composition of embodiment 27, wherein the chemical modification comprises pegylation, acetylation, a d-amino acid, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphatidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, proteolytic processing, phosphorylation, prenylation, racemization, selenoylation, sulfation, and/or transfer-RNA mediated addition of amino acids to protein such as arginylation.

Embodiment 29: The composition of any one of embodiments 25 to 28, wherein the Neu5Gc-Lewis^{a}-binding molecule comprises a peptide having at least 75%, 80%, 85%, 90%, 95%, or 100% homology or identity to a peptide comprising or consisting of the sequence of SEQ ID NO:1 or SEQ ID NO:2.

Embodiment 30: The composition of any one of embodiments 25 to 29, wherein the Neu5Gc-Lewis^{a}-binding peptide comprises a Neu5Gc-Lewis^{a}-binding fragment of MGS5.

Embodiment 31: The composition of any one of embodiments 25 to 30, wherein the Neu5Gc-Lewis^{a}-binding peptide comprises multiple MGS5 peptide moieties, Neu5Gc-Lewis^{a}-binding fragments of MGS5 moieties, and/or Neu5Gc-Lewis^{a}-binding peptides moieties comprising an amino acid sequence that has at least 75%, 80%, 85%, 90%, 95%, or 100% homology or identity to MGS5.

Embodiment 32: The composition of embodiment 31, wherein the multiple peptide moieties are linked to each other through a linker.

Embodiment 33: The composition of embodiment 32, wherein the linker comprises PEG.

Embodiment 34: A chimeric antigen receptor (“CAR”) wherein the CAR targets Neu5Gc-Lewis^{a}, preferably wherein the CAR comprises an anti-Neu5Gc-Lewis^{a} antibody, or a Neu5Gc-Lewis^{a}-binding fragment thereof, or an Neu5Gc-Lewis^{a}-binding peptide in addition to or instead of the antigen receptor (“AR”), wherein the antibody and peptide have been previously determined not to bind any other form of Neu5Gc or any other Sialyl-Lewis^{a}, including free Neu5Gc.

Embodiment 35: A cell comprising the CAR of embodiment 34, preferably wherein the cell is a T lymphocyte (T cell, e.g., CD4+ T cells or CD8+ T cell), cytotoxic lymphocytes (CTL), macrophages or natural killer (NK) cell.

Embodiment 36: The cell of embodiment 35, wherein the Neu5Gc-Lewis^{a}-binding peptide comprises a peptide having at least 75%, 80%, 85%, 90%, 95%, or 100% homology or identity to a peptide comprising or consisting of the sequence of SEQ ID NO:1 or SEQ ID NO:2.

Embodiment 37: A method of treating cancer in a subject in need thereof, comprising administering an anti-cancer therapy to the subject wherein the subject has been selected for treatment because the subject has been diagnosed as having cancer by a method comprising contacting a cell, exosome or tissue sample from the subject with a Neu5Gc-Lewis^{a}-binding molecule and detecting the binding of the molecule to Neu5Gc-Lewis^{a} in the cell, exosome or tissue sample, thereby diagnosing the subject has having cancer.

Embodiment 38: The method of embodiment 37, wherein the Neu5Gc-Lewis^{a}-binding molecule comprises a peptide, antibody, nucleic acid, a carbohydrate, a lipid, a small organic molecule, a lipid particle (e.g., nanoparticle), or an aptamer, wherein the molecule does not bind any other form of Neu5Gc or any other Sialyl-Lewis^{a}, including free Neu5Gc.

Embodiment 39: The method of embodiment 37 or 38, wherein the Neu5Gc-Lewis^{a}-binding molecule comprises a peptide having at least 75%, 80%, 85%, 90%, 95%, or 100% homology or identity to a peptide comprising or consisting of the sequence of SEQ ID NO:1 or SEQ ID NO:2.

Embodiment 40: A method of removing Neu5Gc-Lewis^{a} from milk comprising contacting the milk with a Neu5Gc-Lewis^{a}-binding molecule, preferably comprising a peptide having at least 75%, 80%, 85%, 90%, 95%, or 100% homology or identity to a peptide comprising or consisting of the sequence of SEQ ID NO:1 or SEQ ID NO:2 and then removing the Neu5Gc-Lewis^{a}-binding molecule-bound Neu5Gc-Lewis^{a} from the milk.

Embodiment 41: A method of removing Neu5Gc-Lewis^{a} from cell culture medium or cell culture medium components comprising contacting the medium or medium components with a Neu5Gc-Lewis^{a}-binding molecule comprising a peptide having at least 75%, 80%, 85%, 90%, 95%, or 100% homology or identity to a peptide comprising or consisting of the sequence of SEQ ID NO:1 or SEQ ID NO:2 and then removing the Neu5Gc-Lewis^{a}-binding molecule-bound Neu5Gc-Lewis^{a} from the medium or medium components.

Embodiment 42: A method of reducing stem cell differentiation in vitro comprising culturing/expanding the cells in culture medium previously contacted with a Neu5Gc-Lewis^{a}-binding molecule comprising a peptide having at least 75%, 80%, 85%, 90%, 95%, or 100% homology or identity to a peptide comprising or consisting of the sequence of SEQ ID NO:1 or SEQ ID NO:2 and subsequently depleted of Neu5Gc-Lewis^{a}-binding molecule-bound Neu5Gc-Lewis^{a}.

### BRIEF DESCRIPTION OF DRAWINGS

In order to more clearly illustrate the specific embodiments of the present application or the technical solutions in the prior art, the following will briefly introduce the accompanying drawings that need to be used in the description of the specific embodiments or prior art. Obviously, the accompanying drawings in the following description are some implementations of the present application, and those skilled in the art may obtain other drawings based on these drawings without creative work. The patent or application file contains at least one drawing executed in color. Copies of this patent or patent application publication with color drawings will be provided by the U.S. Patent and Trademark Office upon request and payment of the necessary fee.

FIG. 1: Neu5Gc-Lewis^{a} (R represents additional linked glycosidic side chains that do not compose the core structure of Neu5Gc-Lewis^{a} and/or proteins).

FIG. 2: Glycan array showing MGS5_V1 and MGS5_V2 binding Neu5Gc-Lewis^{a}. Array positive controls (spotted biotin) are depicted at the top and bottom of the array. MGS5_V1 and MGS5_V2 were incubated on the array for 3 hours and binding was visualized using Cy3-Streptavidin.

FIGS. 3A-3F: IHC results showing selective binding of MGS5_V2 to cancer tissue and thus identification of Neu5Gc-Lewis^{a} as a marker for cancer tissue. FIG. 3A: normal/healthy tissue; FIG. 3B: developmental stages of Pancreatic Ductal Adenocarcinoma (PDAC) and metastasized PDAC sites; FIG. 3C, 3D, 3F: PDAC, different magnifications; FIG. 3E: Malignant/cancer tissues.

FIG. 4: Pan02 cells treated with 125 nM of MGS5_V2 with 0.1 U/ml, 0.2 U/ml, and 0.4 U/ml of sialidase (Vibro cholerae) resulted in reduced cellular uptake of MGS5_V2 compared to that of the cells just treated with MGS5_V2. Error bars represent standard error of the mean from three independent experiments. Statistical analysis was done using a one-way ANOVA with multiple comparisons. The asterisks indicate the following P values: * p<0.05; ** p<0.01; **** p<0.0001.

FIG. 5: Structure of AcMGS5 dimer conjugated to Alexa Fluor 647 dye. The copies of the AcMGS5 peptide are linked covalently to the branches (α-amino group and ε-amino groups) of the lysine core through a PEG12 linker. The conjugate to a cargo molecule is formed by reaction of the thiol group (highlighted by arrow) on the dimer core with the appropriate reactive chemical group on the cargo, in this example maleimide AF647.

FIG. 6: Schematic of the use of MGS5 for capture and quantitation of exosomes from serum of tumor bearing mouse.

### DETAILED DESCRIPTION

### Definitions

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “e.g.,” and “ie.,” as used herein, are used merely by way of example, without limitation intended, and should not be construed as referring only those items explicitly enumerated in the specification.

The terms “or more,” “at least,” “more than,” and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000, or more than the stated value. Also included is any greater number or fraction in between.

Conversely, the term “no more than” includes each value less than the stated value. In one embodiment, “no more than 100 monomers” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 monomers. Also included is any lesser number or fraction in between.

The terms “plurality,” “at least two,” “two or more,” “at least second,” and the like, are understood to include but not limited to at least 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000, or more. Also included is any greater number or fraction in between.

Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers, or steps. It is understood that wherever embodiments are described herein with the language “comprising,” otherwise analogous embodiments described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed disclosure.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. In one embodiment, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. In one embodiment, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. In one embodiment, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

A “therapeutically effective amount,” “effective dose,” “effective amount,” or “therapeutically effective dosage” of a therapeutic agent” or “agent” described in the specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms may be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens may be determined empirically by testing in known in vitro or in vivo (e.g., animal model) systems. The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration may occur for several days to years prior to the manifestation of symptoms of an infection. Prophylactic administration may be used, for example, in the chemopreventative treatment of subjects presenting precancerous lesions, those diagnosed with early-stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

The term “combination” refers to either a fixed combination in one dosage unit form, or a combined administration where a compound of the present disclosure and a combination partner (e.g., another drug as explained below, also referred to as “therapeutic agent” or “agent”) may be administered independently at the same time or separately within time intervals, especially where these time intervals allow that the combination partners show a cooperative, e.g., synergistic effect. The single components may be packaged in a kit or separately. One or both of components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

A “patient” or a “subject” as used herein includes any human who is afflicted with a cancer or any disorder, particularly one that increases the level of the biomarker Neu5Gc-Lewis^{a}. The terms “subject” and “patient” are used interchangeably herein.

As used herein, the term “epitope” refers to an antigenic determinant that interacts with (is bound by) a specific antigen binding site in the variable region of an antibody molecule (the paratope). A single antigen (such as, but not limited to, a polypeptide) may have more than one epitope. Thus, different antibodies may bind to different epitopes on an antigen and may have different biological effects depending on which epitope is bound. The term “epitope” also refers to a site on an antigen to which B and/or T cells respond. It also refers to a region of an antigen that is bound by an antibody. Epitopes may be defined as a structural epitope (the portion of the antigenic determinant that is contacted by the CDR loops of an antibody) or a functional epitope (a subset of a structural epitope comprising those energetic residues centrally located in the structural epitope and directly contribute to the affinity of the antibody-epitope interaction). Epitopes may become immunologically available after fragmentation or denaturation of an antigen (a cryptotope). Epitopes may be linear or conformational (composed of non-linear amino acids brought together in a folded three-dimensional structure). Epitopes may include residues that are chemically active surface groupings of molecules such as amino acids, sugar side chains, phosphoryl groups, or sulfonyl groups, and may have specific three-dimensional structural characteristics, and/or specific charge characteristics. An epitope typically includes at least 3 to 15 amino acids.

The term “antibody” is used herein in the broadest sense and encompasses various antibody structures and antibody fragments so long as they exhibit the desired antigen-binding activity and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins may be assigned to different classes. There are five major classes of immunoglobulins: IgA, IgD, IgE, IgG, and IgM, and several of these may be further divided into subclasses (isotypes, e.g., IgG1, IgG2, IgG3, IgG4, IgAQ1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. Examples of antibody structures include, but are not limited to, monoclonal antibodies, recombinantly produced antibodies, monospecific antibodies, multi-specific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affibodies, Fab, Fab′, F(ab′)2, and Fv fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”). In one embodiment, the antibody has been previously approved for clinical use by a regulatory agency.

A “humanized” antibody refers to a chimeric antibody comprising amino acid residues from non-human HVRs and amino acid residues from human frame regions (FRs). In certain embodiments, a humanized antibody will comprise substantially all of at least one, and typically two, variable domains, in which all or substantially all of the hypervariable regions (HVRs) (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and HVRs. A single VH or VL domain may be sufficient to confer antigen-binding specificity. Furthermore, antibodies that bind a particular antigen may be isolated using a VH or VL domain from an antibody that binds to the antigen to screen a library of complementary VL or VH domains, respectively. See, e.g., Portolano et al., J. Immunol. 150: 880-887 (1993); Clarkson et al., Nature 352: 624-628 (1991).

The term “antigen-binding fragment” refers to a molecule other than an intact antibody, such molecule comprises a portion of the intact antibody and binds to the antigen to which the intact antibody binds. Examples of antigen-binding fragments include, but are not limited to, Fv, Fab, Fab′, Fab′-SH, F(ab′) 2, diabodies, dAb, linear antibody, single-chain antibodies (e.g., scFv); single-domain antibodies; antigen-binding fragments of bivalent or bispecific antibodies; camelid antibodies; single domain antibodies, maxibodies, minibodies, nanobodies, intrabodies, diabodies, triabodies, tetrabodies, v-NAR and bis-scFv, and other fragments with desired antigen (e.g., Neu5Gc-syalyl Lewis-a)-binding ability.

The term “antigen-binding site” refers to the part of an antibody molecule that comprises determinants that form an interface that binds to a polypeptide, or an epitope thereof. With respect to proteins (or protein mimetics), the antigen-binding site typically includes one or more loops (e.g., of at least four amino acids or amino acid mimics) that form an interface that binds to a polypeptide. Typically, the antigen-binding site of an antibody molecule includes at least one or two CDRs and/or hypervariable loops, or more typically at least three, four, five or six CDRs and/or hypervariable loops.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as LI, L2 and L3 or H1, H2 and H3 where the “L” and the “H” designate the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9: 133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDR.

As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a KD (affinity) for binding the target of at least about 10-4M, 10-5M, 10-6M, 10-7M, 10-8M, 10-9M, 10-10M, 10-11M, 10-12M, 10-13M, or less, preferably as measured by the method of the Examples (biolayer interferometry (BLI).

The term “linker” refers to a chemical moiety comprising a covalent bond or a chain of atoms that covalently attaches one molecule to another (e.g., a peptide to another peptide). In various embodiments, linkers include a divalent radical such as an alkyldiyl, an aryldiyl, a heteroaryldiyl, moieties such as: —(CR2)nO(CR2)n-, repeating units of alkyloxy (e.g., polyethylenoxy, PEG, polymethyleneoxy) and alkylamino (e.g., polyethyleneamino); and diacid ester and amides including succinate, succinamide, diglycolate, malonate, and caproamide. In various embodiments, linkers may comprise one or more amino acid residues, such as valine, phenylalanine, Cys, lysine, and homolysine.

The term “synthetic” is generally used herein to refer to compounds or molecules, e.g., compounds described herein, that are not naturally occurring.

As used herein, the term “polypeptide” refers to polymers of amino acids. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide may be isolated from natural sources, may be produced by recombinant techniques from a eukaryotic or prokaryotic host, or may be a product of synthetic procedures. In some embodiments, the polypeptide is greater than 50 amino acids in length.

As used herein, “peptide” is less than or equal to 50 amino acids long, e.g., about 5, 10, 15, 20, 25, 30, 35, 40, 45, or 50 amino acids long.

As used herein, the term “nucleic acid” refers to a polynucleotide sequence, or fragment thereof. A nucleic acid may comprise nucleotides. A nucleic acid may be exogenous or endogenous to a cell. A nucleic acid may exist in a cell-free environment. A nucleic acid may be a gene or fragment thereof. A nucleic acid may be DNA. A nucleic acid may be RNA. A nucleic acid may comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” may be used interchangeably. A nucleic acid may comprise one or more modifications (e.g., a base modification, a backbone modification), to provide the nucleic acid with a new or enhanced feature (e.g., improved stability). A nucleic acid may comprise a nucleic acid affinity tag.

A nucleic acid may comprise a nucleic acid mimetic. The term “mimetic” may be intended to include polynucleotides wherein only the furanose ring or both the furanose ring and the internucleotide linkage are replaced with non-furanose groups, replacement of only the furanose ring may also be referred as being a sugar surrogate. The heterocyclic base moiety or a modified heterocyclic base moiety may be maintained for hybridization with an appropriate target nucleic acid. One such nucleic acid may be a peptide nucleic acid (PNA). In a PNA, the sugar-backbone of a polynucleotide may be replaced with an amide containing backbone, in particular with an aminoethylglycine backbone. The nucleotides may be retained and are bound directly or indirectly to aza nitrogen atoms of the amide portion of the backbone. The backbone in PNA compounds may comprise two or more linked aminoethylglycine units which gives PNA an amide containing backbone. The heterocyclic base moieties may be bound directly or indirectly to aza nitrogen atoms of the amide portion of the backbone.

The term “aptamer” refers to biomolecules that may be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Aptamers may be peptides. Aptamers may also be nucleic acids. Nucleic acid aptamers may for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and Hicke B J, Stephens A W. “Escort aptamers: a delivery service for diagnosis and therapy.” J Clin Invest 2000, 106:923-928). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4). In some embodiments, “a nucleic acid aptamer” refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that may covalently bind to or crosslink with a target molecule. The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “affinity” as used herein refers to the strength of the sum of all noncovalent interactions between a single binding site of a molecule (e.g., an antibody) and its binding partner (e.g., an antigen). Unless otherwise indicated, as used herein, “binding affinity” refers to the intrinsic binding affinity which reflects a 1:1 interaction between members of a binding pair (e.g., an antibody and an antigen). The affinity of molecule X for its partner Y is generally expressed by the dissociation constant (KD). Methods for determining binding affinity are known in the art, including surface plasmon resonance (e.g., SPR, BIACORE) or similar techniques (e.g., ForteBio; biolayer interferometry (BLI)).

As used herein the term “associated” or “associated with” may mean that Neu5Gc-Lewis^{a} is identifiable as being localized to the cell, exosome or tissue at a point in time.

The term “label” refers to detectable moieties, such as those whose molecular size is sufficient to induce detectable modifications in their physical and/or chemical properties; such detection may be achieved by optical methods (such as fluorescence, diffraction, surface plasmon resonance, surface variation and contact variation angles) or Physical methods such as atomic force spectroscopy and tunneling effect, and electron-dense substances, such as radioactive molecules (e.g. ³²P, ³S or ¹²⁵I). In one embodiment, “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” may generate a measurable signal and may be covalently or noncovalently joined to a ligand. The term “ligand” refers to the molecule to which the label binds to and which binds or complexes with directly or indirectly. In some embodiments, the ligand is Neu5Gc-Lewis^{a}, a molecular form or fragment thereof. In certain embodiments, in which the Neu5Gc-Lewis^{a} is to be evaluated, the ligand may be a peptide or an antibody. In certain embodiments, the ligand is MGS5.

By “biomarker” as used herein is meant a single molecule (e.g., Neu5Gc-Lewis^{a}), the levels or relative levels or ratios of which in a cell, exosome or tissue in a subject significantly change (either in an increased or decreased manner) from the level or relative levels present in a subject having one physical condition or disease or disease stage representative of another physical condition or disease stage, including “normal” or non-diseased stage. “Reference standard” as used herein refers to the source of the reference biomarker levels. The “reference standard” is preferably provided by using the same assay technique as is used for measurement of the subject's biomarker levels in the reference subject or population, to avoid any error in standardization. The reference standard is, alternatively, a numerical value, a predetermined cut point, a mean, an average, a numerical mean or range of numerical means, a numerical pattern, a ratio, a graphical pattern or a marker abundance profile or marker level profile derived from the same biomarker or biomarkers in a reference subject or reference population. “Reference subject” or “Reference Population” defines the source of the reference standard. In one embodiment, the reference is a human subject or a population of subjects having no cancer or other disease, i.e., healthy controls or negative controls. In yet another embodiment, the reference is a human subject or population of subjects with one or more clinical indicators of cancer or other disease, but who did not develop the cancer or other disease. In still other embodiments of methods described herein, the reference is obtained from the same test subject who provided a temporally earlier biological sample. That sample may be pre- or post-therapy or pre- or post-surgery.

“Sample” or “biological sample” as used herein means any biological fluid or tissue that may or may not comprise Neu5Gc-Lewis^{a}. The most suitable samples for use in the methods and with the compositions are samples which require minimal invasion for testing, e.g., blood samples, including cerebrospinal fluid, serum, plasma, whole blood, and circulating tumor cells. It is also anticipated that other biological fluids, such as saliva or urine, vaginal or cervical secretions, and ascites fluids or peritoneal fluid may be similarly evaluated by the methods described herein. Also, circulating tumor cells or fluids containing them are also suitable samples for evaluation in certain embodiments of this disclosure. The samples may include biopsy tissue, tumor tissue, surgical tissue, circulating tumor cells, or other tissue. Such samples may further be diluted with saline, buffer or a physiologically acceptable diluent. Alternatively, such samples are concentrated by conventional means. In certain embodiments, e.g., those in which Neu5Gc-Lewis^{a} levels are desired to be evaluated, the samples may include biopsy tissue, surgical tissue, circulating tumor cells, or other tissue. In one embodiment, the sample is a tumor secretome, i.e., any fluid or medium containing the proteins secreted from the tumor. These shed proteins may be unassociated, associated with other biological molecules, or enclosed in a lipid membrane such as an exosome. In another embodiment, the sample is plasma. In some embodiments, the sample is a tissue sample, for example, tumor tissue, and may be fresh, frozen, or archival paraffin embedded tissue.

A biological sample is typically obtained from a eukaryotic organism, most preferably a mammal, or a bird, reptile, or fish. Indeed, a “subject” which may be subjected to the methods described herein may be any mammalian animals including human, dog, cat, cattle, goat, pig, swine, sheep and monkey; or a bird; reptile; or fish. Preferably, a subject is a human being; a human subject may be known as a “patient” By “obtaining a biological sample,” it is herein meant to obtain a biological sample for use in methods described in this disclosure. Most often, this will be done by removing a sample of cells from an animal but may also be accomplished by using previously isolated cells (e.g., isolated by another person, at another time, and/or for another purpose), or by performing the methods of the disclosure in vivo. Archival tissues, having treatment or outcome history, will be particularly useful.

The degree of change (or differential expression) in Neu5Gc-Lewis^{a} level may vary with each individual and is subject to variation with each population. For example, in one embodiment, a large change, e.g., 2-3 fold increase or decrease in Neu5Gc-Lewis^{a} levels, is statistically significant. In another embodiment, a larger relative change of 10-fold increase or more is statistically significant. The degree of change in Neu5Gc-Lewis^{a} levels varies with the condition, such as type of cancer and with the size or spread of the cancer or solid tumor. The degree of change also varies with the immune response of the individual and is subject to variation with each individual. For example, in one embodiment of this disclosure, a change at or greater than a 1.2 fold increase or decrease in Neu5Gc-Lewis^{a} levels, is statistically significant. In another embodiment, a larger change, e.g., at or greater than a 1.5 fold, greater than 1.7 fold or greater than 2.0 fold increase or a decrease in the level of Neu5Gc-Lewis^{a} is statistically significant. Still alternatively, if Neu5Gc-Lewis^{a} level is significantly increased in biological samples which normally do not contain measurable levels of Neu5Gc-Lewis^{a}, such increase in Neu5Gc-Lewis^{a} level may alone be statistically significant. In one embodiment, the chance or different is significant when the fold-change in Neu5Gc-Lewis^{a} levels compared to control level is at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold different in the sample versus the control or in one sample versus another. The differential Neu5Gc-Lewis^{a} levels takes into account both increase and decreased levels. Neu5Gc-Lewis^{a} may be considered up or down-regulated if the differential expression meets a statistical threshold, a fold-change threshold, or both. For example, the criteria for identifying differential expression may comprise both a p-value of 0.001 and fold change of at least 1.2-fold (up or down). Conversely, if Neu5Gc-Lewis^{a} level is normally decreased or not significantly measurable in certain biological samples which normally do contain measurable Neu5Gc-Lewis^{a} levels, such decrease in Neu5Gc-Lewis^{a} may alone be statistically significant. When Neu5Gc-Lewis^{a} levels are being evaluated or measured by immunohistochemistry, the presence versus absence of a signal indicating the presence vs absence of Neu5Gc-Lewis^{a} is considered a significant change and a diagnostic of cancer (when present). One of ordinary skill in the art is familiar with methods of relative quantification of immunohistochemistry signals for the change to be considered significant.

The term “determining the level of Neu5Gc-Lewis^{a} in a sample, control or reference, as described herein, shall refer to the quantification of the presence of Neu5Gc-Lewis^{a} in the tested sample. The level may be quantitative or qualitative. For example, the concentration of Neu5Gc-Lewis^{a} in said samples may be directly quantified via measuring the amount of Neu5Gc-Lewis^{a} as present in the tested sample. Moreover, it is also possible to quantify the amount of Neu5Gc-Lewis^{a}, for example by mass spectrometry. How to determine the level of Neu5Gc-Lewis^{a} is well known to the skilled artisan. The present disclosure shall not be restricted to any particular method for determining the level of Neu5Gc-Lewis^{a} but shall encompass all means that allow for a quantification, or estimation, of the level of Neu5Gc-Lewis^{a}, either directly or indirectly. A quantitative “Level” in the context of the present disclosure is therefore a parameter describing the absolute amount of Neu5Gc-Lewis^{a}, for example as absolute weight, volume, or molar amounts; or alternatively “level” pertains to the relative amounts, and preferably to the concentration of Neu5Gc-Lewis^{a} in the tested sample, for example in mol/1, g/1, g/mol. A qualitative “level” may be evaluated by immunohistochemistry, fluorescence activated cell sorting, and the like.

A “diagnosis” or the term “diagnostic” in the context of the present disclosure means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “monitoring of a disease, condition or disorder” shall mean for the purpose of the present disclosure to observe disease progression in a patient who receives a therapy. In other words, the patient during the therapy is regularly monitored for the effect of the applied therapy, which allows the medical practitioner to estimate at an early stage during the therapy whether the prescribed treatment is effective or not, and, therefore, to adjust the treatment regime accordingly.

The term “prognosis” refers to a forecast as to the probable outcome of the disease as well as the prospect of recovery from the disease as indicated by the nature and symptoms of the case. Accordingly, a negative or poor prognosis is defined by a lower post-treatment survival term or survival rate. Conversely, a positive or good prognosis is defined by an elevated post-treatment survival term or survival rate. Usually, prognosis is provided as the time of progression free survival (PFS) or overall survival (OS).

### I. NEU5GC-SIALYL LEWIS^{A}OR NEU5GC-LEWIS^{A} AS A BIOMARKER

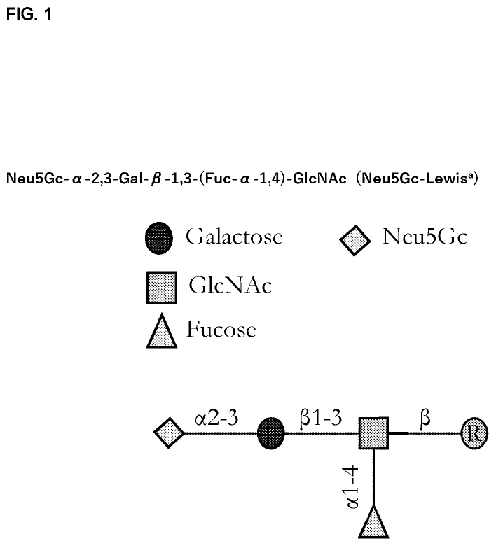

The monosaccharide N-glycolyl neuraminic acid (Neu5Gc) is among the most common sialic acid forms in nature. Lewis a (Le^{a}) antigen is a blood group antigen and it is a carbohydrate found in glycolipids and glycoproteins on the surface of various cells. Although humans are not able to synthesize Neu5Gc, N-glycolylneuraminic acid (Neu5Gc)-Lewis^{a} is known as a dietary-derived glycan. The tetrasaccharide structures Siaα2,3Galβ1,3(Fucα1,4)GlcNAc constitute the epitopes of the carbohydrate antigens sialyl-Lewis a (sLe^{a}). FIG. 1. For purposes of this disclosure, references to Neu5Gc-Lewis^{a} are more specifically references to Neu5Gc-α-2,3-Gal-β-1,3-(Fuc-α-1,4)-GlcNAc-β.

It is known that Neu5Gc itself is metabolically incorporated into human tissues from dietary sources (particularly red meat) and detected at even higher levels in some human cancers. But it was not known that the Neu5Gc-Lewis^{a} is a more specific marker for cancer cells and tissues. The disclosure provides evidence that Neu5Gc-Lewis^{a} is present on the cell surface and extracellular matrix of cancer tissues and not in non-diseased tissues or healthy tissues such as brain, liver, lymph node, and pancreas. In other words, the disclosure provides that Neu5Gc-Lewis^{a} is a biomarker for cancer. Neu5Gc-Lewis^{a} is superior to other forms of Neu5Gc as a cancer biomarker and cancer-target, at least because those other forms of Neu5Gc have been found to be present in some healthy tissues. Accordingly, in one embodiment, the disclosure provides methods of diagnosing cancer and targeting cancer tissues with a variety of molecules that bind Neu5Gc-Lewis^{a}.

### II. DIAGNOSIS

In one embodiment, the presence of Neu5Gc-Lewis^{a} in a cell, exosome or tissue may be used in the diagnosis or prognosis of cancer. Accordingly, in one embodiment, the disclosure provides a method of detecting Neu5Gc-Lewis^{a} on the surface of a cell or on a tissue, comprising (a) obtaining or having obtained a cell or tissue; (b) detecting whether the Neu5Gc-Lewis^{a} is present on the surface of the cell by contacting the cell with a molecule previously known to bind Neu5Gc-Lewis^{a}, also referred to henceforth as a Neu5Gc-Lewis^{a}-binding molecule, and detecting binding between Neu5Gc-Lewis^{a} and the Neu5Gc-Lewis^{a}-binding molecule on the cell surface. In one embodiment, the disclosure provides a method of detecting Neu5Gc-Lewis^{a} in the extracellular matrix (ECM) of a cell or tissue comprising (a) obtaining or having obtained a cell or tissue; (b) detecting whether the Neu5Gc-Lewis^{a} is present on the extracellular matrix of the cell or tissue by contacting the cell or tissue with a Neu5Gc-Lewis^{a}-binding molecule, and detecting binding between Neu5Gc-Lewis^{a} and the Neu5Gc-Lewis^{a}-binding molecule on the extracellular matrix of the cell or tissue. In some embodiments, the cell or tissue is from a subject having or at risk of cancer. In some embodiments, the method further comprises diagnosing the subject with cancer upon detection of Neu5Gc-Lewis^{a} on the extracellular matrix of the cell or tissue. In some embodiments, the method further comprises administering a cancer treatment to the subject diagnosed as having cancer.

In one embodiment, the disclosure provides a method of detecting Neu5Gc-Lewis^{a} in an exosome released from a cell or tissue comprising (a) obtaining or having obtained a sample comprising an exosome released from a cell or tissue; (b) detecting whether the Neu5Gc-Lewis^{a} is present on the exosome by contacting the exosome with a Neu5Gc-Lewis^{a}-binding molecule, and detecting binding between Neu5Gc-Lewis^{a} and the Neu5Gc-Lewis^{a}-binding molecule on the exosome. In some embodiments, the sample is from a subject having or at risk of cancer. In some embodiments, the method further comprises diagnosing the subject with cancer upon detection of Neu5Gc-Lewis^{a} on an exosome in the sample from the subject. In some embodiments, the method further comprises administering a cancer treatment to the subject diagnosed as having cancer.

In one embodiment, the cell is a cancer cell. In one embodiment, the tissue is normal tissue. In one embodiment, the tissue is diseased tissue. In one embodiment, the tissue is suspected of comprising cancer tissue or is known to comprise cancer tissue. In one embodiment, the tissue comprises blood vessels. In one embodiment, the blood vessels are arteries.

In one embodiment, the presence of Neu5Gc-Lewis^{a} in either the cell surface, exosome and/or ECM of the cell or tissue identifies or diagnoses the cell or tissue as cancer tissue. In one embodiment, a significant increase in the level of Neu5Gc-Lewis^{a} in the cell, exosome or tissue, relative to a healthy cell, exosome from a healthy cell or healthy tissue counterpart, identifies the diagnoses the cell or tissue as cancer tissue. In one embodiment, the presence of Neu5Gc-Lewis^{a} in the cell surface, exosome and/or ECM of the tissue comprising blood vessels identifies or diagnoses the Neu5Gc-Lewis^{a}-positive area as atherosclerotic tissue.

In one embodiment, the disclosure provides a method of detecting extracellular or cell-tissue-bound Neu5Gc-Lewis^{a} in a subject, comprising (a) obtaining or having obtained a sample of solid tissue, blood, serum, plasma, lavage, urine, milk, CSF, or other tissue or fluid from the subject; and (b) detecting whether Neu5Gc-Lewis^{a} is present in the sample by contacting the sample with a Neu5Gc-Lewis^{a} binding molecule, preferably an antibody, peptide, or aptamer, wherein the molecule does not bind any other form of Neu5Gc or any other Sialyl-Lewis^{a}, including free Neu5Gc, and detecting binding between the Neu5Gc-Lewis^{a}-binding molecule and the Neu5Gc-Lewis^{a} in the sample.

In one embodiment, the disclosure provides a method of detecting whether a disease is associated with the presence of Neu5Gc-Lewis^{a}, comprising (a) obtaining or having obtained diseased tissue from a subject and (b) contacting the diseased tissue with a Neu5Gc-Lewis^{a}-binding molecule and detecting the binding between the Neu5Gc-Lewis^{a}-binding molecule, preferably an antibody, peptide, or aptamer, and the Neu5Gc-Lewis^{a} in the diseased tissue, wherein the molecule does not bind to any other form of Neu5Gc or any other Sialyl-Lewis^{a}, including free Neu5Gc.

In one embodiment, the disclosure provides a method of detecting atherosclerosis in a tissue, comprising contacting the tissue with a Neu5Gc-Lewis^{a} binding molecule, preferably an antibody, peptide, or aptamer, and detecting the binding between the Neu5Gc-Lewis^{a} binding molecule and the Neu5Gc-Lewis^{a} in the vessel wall in the tissue, thereby detecting atherosclerosis in the vessel wall, wherein the molecule does not bind any other form of Neu5Gc or any other Sialyl-Lewis^{a}, including free Neu5Gc.

### III. CANCERS

The cancer may be any cancer. In one embodiment, the cancer comprises pancreatic cancer (including ductal adenocarcinoma); breast cancer (including infiltrating duct carcinoma, infiltrating lobular carcinoma, medullary carcinoma), colon cancer (including adenocarcinoma), endometrial cancer (including adenocarcinoma), liver cancer (including hepatocellular carcinoma and cholangiocarcinoma); lung cancer (including squamous cell carcinoma), ovarian cancer (including serous surface papillary carcinoma, poorly differentiated; serous adenocarcinoma, moderately differentiated, serous adenocarcinoma); prostate cancer (including adenocarcinoma), stomach cancer (including adenocarcinoma signet ring cell type); and/or urinary bladder cancer (transitional cell carcinoma).

In one embodiment, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer comprises at least one tumor type selected from the group consisting of pancreatic cancer, breast cancer, biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, and uterine sarcoma. Subtypes of these cancers are also within the scope of the disclosure.

In one embodiment, the term “pancreatic cancer” shall encompass benign or malignant forms of pancreatic cancer, as well as any particular type of cancer arising from cells of the pancreas (e.g., duct cell carcinoma, acinar cell carcinoma, papillary carcinoma, adenosquamous carcinoma, undifferentiated carcinoma, mucinous carcinoma, giant cell carcinoma, mixed type pancreatic cancer, small cell carcinoma, cystadenocarcinoma, unclassified pancreatic cancers, pancreatoblastoma, and papillary-cystic neoplasm, and the like.)

In some embodiments, (i) the subject has not yet been determined to have a cancer, (ii) the subject has not yet been determined to harbor a cancer cell, or and/or (iii) the subject does not exhibit, or has not exhibited, a symptom associated with a cancer. In some embodiments, the presence of cancer is detected at a time period when the subject has not been diagnosed with a stage II cancer, has not been diagnosed with a stage I cancer, has not had a biopsy to confirm abnormal cellular growth, has not had a biopsy to confirm the presence of a tumor, has not undergone a diagnostic scan to detect a cancer, or any combination thereof. In some embodiments, said subject is a member of a population with a low risk, a medium risk, or a high risk of having the cancer based on one or more of the following factors: environmental factors, age, sex, medical history, medications, genetic factors, biochemical factors, biophysical factors, physiological factors, and/or occupational factors.

### IV. METHODS OF TREATMENT COMBINED WITH DIAGNOSIS, PROGNOSIS, AND THERAPEUTIC INTERVENTION OR STRATIFICATION

In one embodiment, the disclosure provides methods of treatment according to the discovery that Neu5Gc-Lewis^{a} is a biomarker for cancer. Accordingly, in one embodiment, the disclosure provides a method of cancer treatment in a subject in need thereof, whereby the subject is identified as having cancer and/or selected for treatment using the methods of the disclosure (i.e., detection of the presence of Neu5Gc-Lewis^{a} in a subject's tissue or body fluid and subsequently administered a cancer treatment). Non-limiting examples of cancers and cancer treatments are provided above. In some embodiments, the subject has previously received another treatment. In some embodiments, the levels of Neu5Gc-Lewis^{a} in a subject's tissue or body fluid are measured before, during, and after receiving treatment and are used to monitor treatment response. In some embodiments, the subject is said to have responded to the treatment when the circulating or tissue-bound levels of Neu5Gc-Lewis^{a} in a subject's tissue or body fluid decrease after treatment. In some embodiments, the decrease is statistically significant.

Accordingly, in one embodiment, the disclosure provides a method for the prognosis, diagnosis, monitoring of a disease, and/or stratification of a condition or disorder in a subject, comprising the steps of (a) Providing a biological sample of said subject, (b) Determining the level of Neu5Gc-Lewis^{a} in a cell and/or extracellular matrix or tissue in said biological sample, (c) Determining the level of Neu5Gc-Lewis^{a} in second sample of the same kind obtained from the same subject, and (d) Comparing the determined level, in steps (b) and (c) with a reference sample or a reference value, and/or to each other, wherein a differential level of Neu5Gc-Lewis^{a} is indicative of disease presence and/or disease progression or regression in said subject, when samples of steps b) and c) are biological samples from normal vs disease tissue, or different stages of treatment or before treatment, respectively. In one embodiment, the disease is cancer. In some embodiments, the disease therapy is adjusted based on the level of Neu5Gc-Lewis^{a} in the samples. In one embodiment, the therapy is anti-cancer therapy. In one embodiment, the therapy is radiation. In one embodiment, the therapy is a therapeutic agent. In one embodiment, the therapy is combinations of the same. Non-limiting examples of therapeutic agents are described elsewhere in the application.

In one embodiment, detection of extracellular or cell-tissue-bound Neu5Gc-Lewis^{a} may discriminate cancer patients from cancer-free individuals and/or monitor cancer progression in the subject. In one embodiment, the efficacy of a treatment may be monitored by measuring the area of the diseased tissue in the subject that binds to the Neu5Gc-Lewis^{a} binding molecule and/or the level of Neu5Gc-Lewis^{a} in the tissue or body fluid, before and after exposure of the subject to the treatment.

Accordingly, in one embodiment, the disclosure provides a method for monitoring the effectiveness of an anti-cancer therapy comprising determining the level of Neu5Gc-Lewis^{a} in a sample from a subject treated with an anti-cancer therapy in accordance with the embodiments. In some embodiments, if the levels of the Neu5Gc-Lewis^{a} are significantly elevated as compared to a reference level, the subject is in need of additional anti-cancer therapy. Conversely, if the level of Neu5Gc-Lewis^{a} is not elevated as compared to a reference level, the subject is not in need of additional anti-cancer therapy. Thus, in certain embodiments, a method is provided for monitoring the effectiveness of an anti-cancer therapy comprising (a) determining the levels of a Neu5Gc-Lewis^{a} in a sample from a subject before and after treatment with an anti-cancer therapy; and (b) identifying the subject as responsive to the therapy or not responsive to the therapy based on the change in levels of Neu5Gc-Lewis^{a}. For example, the level of a Neu5Gc-Lewis^{a} in a responsive subject should be reduced after therapy. In further embodiments, a subject identified as not responsive to the first anticancer therapy may be administered a second anti-cancer therapy.

The skilled artisan will understand that numerous methods may be used to select a threshold or reference value for a particular marker (e.g., Neu5Gc-Lewis^{a}). In diagnostic embodiments, a threshold value may be obtained by performing the assay method on samples obtained from a population of patients having, for example, a certain type of cancer, and from a second population of subjects that do not have cancer. Alternatively, a threshold value may be obtained by performing the assay method on samples obtained from a population of patients having, for example, an aggressive type of cancer associated with a reduced time of progression free survival (PFS) and/or overall survival (OS), and from a second population of subjects that do have a rather mild form of cancer associated with an increased time of progression free survival (PFS) and/or overall survival (OS).

In the context of the above method, an increase or a decrease of a differential level, of the biomarker Neu5Gc-Lewis^{a} may be indicative of a reduced time of progression free survival (PFS) and/or overall survival (OS). If decreased levels of the Neu5Gc-Lewis^{a} are associated with poor prognosis, then also a decrease of Neu5Gc-Lewis^{a} in said sample may be indicative of a reduced time of progression free survival (PFS) and/or overall survival (OS).

In one embodiment, for prognostic or treatment monitoring applications, a population of patients, all of which have, for example, pancreatic cancer, may be followed for the time period of interest (e.g., six months following diagnosis or treatment, respectively), and then dividing the population into multiple groups, for example two groups: a first group of subjects that progresses to an endpoint (e.g., recurrence of disease, and/or death); and a second group of subjects that did not progress to the end point (e.g., no recurrence of disease, and/or no death). These are used to establish “low risk” and “high risk” population values for the marker(s) measured, respectively. Other suitable endpoints include, but are not limited to, 5-year mortality rates or progression to metastatic disease.

In one embodiment, once these groups are established, one or more thresholds may be selected that provide an acceptable ability to predict prognostic risk, diagnosis, treatment success, etc. In practice, Receiver Operating Characteristic curves, or “ROC” curves, are typically calculated by plotting the value of a variable versus its relative frequency in two populations (called arbitrarily “disease” and “normal” or “low risk” and “high risk”, for example). For any particular marker, a distribution of marker level for subjects with and without a disease may overlap. Under such conditions, a test does not absolutely distinguish “disease” and “normal” with 100% accuracy, and the area of overlap indicates where the test cannot distinguish “disease” and “normal.” A threshold is selected, above which (or below which, depending on how a marker changes with the disease) the test is considered to be “positive” and below which the test is considered to be “negative.” The area under the ROC curve (AUC) is a measure of the probability that the perceived measurement may allow correct identification of a condition.

In one embodiment, thresholds may be established by obtaining an earlier marker result from the same patient, to which later results may be compared. In some embodiments, the individuals act as their own “control group.” In markers that increase with disease severity or prognostic risk, an increase over time in the same patient may indicate a worsening of disease or a failure of a treatment regimen, while a decrease over time may indicate remission of disease or success of a treatment regimen.

In some embodiments, multiple thresholds or reference values may be determined. This may be the case in so-called “tertile,” “quartile,” or “quintile” analyses. In these methods, the “disease” and “normal” groups (or “low risk” and “high risk”) groups may be considered together as a single population, and are divided into 3, 4, or 5 (or more) “bins” having equal numbers of individuals. The boundary between two of these “bins” may be considered “thresholds.” A risk (of a particular prognosis or diagnosis for example) may be assigned based on which “bin” a test subject falls into.

The term “stratification” for the purposes of this disclosure shall refer to the advantage that the methods according to the disclosure render decisions for the treatment and therapy of the patient possible, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof. Particularly with regard to pancreatic or breast cancer, “stratification” means in this context a classification of a pancreatic or breast cancer disease of an individual patient with regard of the metastatic status, or the presence or absence of circulating tumor cells. Also, the term “stratification” covers, in particular, the risk stratification with the prognosis of an outcome of a negative health event.

### V. OTHER NON-LIMITING EXEMPLARY APPLICATIONS OF THE MOLECULES AND METHODS OF THE DISCLOSURE

The disclosure provides that the presence of Neu5Gc-Lewis^{a} in a cell, tissue, or fluid, may be indicative of the presence of diseases other than cancer. In one embodiment, the disease is selected from infectious diseases, neurological disease, neurodegenerative disease, kidney disease, liver disease, heart disease, lung disease, gastric disease, ocular disease, bone disease, obesity, hematologic malignancies, autoimmune disorders, hypercholesterolemia, asthma, osteoporosis, inflammatory bowel disease, allograft rejection, drug reversal. The detection of Neu5Gc-Lewis^{a} in the diseased tissue or body fluid but not in the normal tissue or body fluid counterpart is an indication that Neu5Gc-Lewis^{a} may be used as a biomarker in the detection of the disease. In one embodiment, a subject is identified as having one or more of those diseases using the methods of the disclosure and subsequently administered a therapy for the disease.

In one embodiment, the disclosure provides a method of removing Neu5Gc-Lewis^{a} from milk comprising contacting the milk with Neu5Gc-Lewis^{a}-binding molecule and then removing the Neu5Gc-Lewis^{a}-binding molecule-bound Neu5Gc-Lewis^{a} from the milk. In one embodiment, the Neu5Gc-Lewis^{a}-binding molecule comprises MGS5.

In one embodiment, the disclosure provides a method of removing Neu5Gc-Lewis^{a} from cell culture medium or cell culture medium components comprising contacting the medium of medium components with Neu5Gc-Lewis^{a}-binding molecule and then removing the Neu5Gc-Lewis^{a}-binding molecule-bound Neu5Gc-Lewis^{a} from the medium. In one embodiment, the Neu5Gc-Lewis^{a}-binding molecule comprises MGS5. In one embodiment, this method may be used in improving the quality and production of recombinantly produced proteins, including antibodies, where the presence of Neu5Gc is undesirable.

A method of reducing stem cell differentiation in vitro comprising culturing/expanding the cells in culture medium previously contacted with Neu5Gc-Lewis^{a}-binding molecule and subsequently depleted of Neu5Gc-Lewis^{a}-binding molecule-bound Neu5Gc-Lewis^{a}. In one embodiment, the Neu5Gc-Lewis^{a}-binding molecule comprises MGS5.

### VI. NEU5GC-LEWIS^{A}-BINDING MOLECULES

A Neu5Gc-Lewis^{a}-binding molecule of the disclosure may be any molecule capable of binding Neu5Gc-Lewis^{a}. In some embodiments, the Neu5GC-Lewis^{a}-binding molecule is specific for Neu5Gc-Lewis^{a} because it does not bind any other form of Neu5Gc or any other Sialyl-Lewis^{a}, including free Neu5Gc.

In one embodiment, the Neu5Gc-Lewis^{a}-binding molecule comprises a peptide, antibody, nucleic acid, a carbohydrate, a lipid, a small organic molecule, a lipid particle (e.g., nanoparticle), or an aptamer. In one embodiment, the aptamer may be a nucleic acid-based aptamer or a peptide-based aptamer.

In one embodiment, the antibody is selected from monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affibodies, Fab, Fab′, F(ab′)2, and Fv fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics.”

In one embodiment, the Neu5Gc-Lewis^{a}-binding molecule comprises a peptide. In one embodiment, the peptide comprises a peptide that has at least 75%, 80%, 85%, 90%, 95%, or 100% homology or identity to a peptide comprising or consisting of the sequence of SEQ ID NO:1 or SEQ ID NO:2.
SEQ ID NO:1 is: LQWRRNFGVWARYRL
SEQ ID NO:2 is: LQWRRDDNVHNFGVWARYRL

For purposes of this disclosure, the peptide of SEQ ID NO:1 is also referred to as MGS5_V2 and the peptide of SEQ ID NO:2 is also referred to as MGS5_V1. The term MGS5 relates to MGS5-V1 and MGS5-V2, indiscriminately.

Accordingly, in one embodiment, the methods of the disclosure may be practiced with a peptide that has at least 75%, 80%, 85%, 90%, 95%, or 100% homology or identity to a peptide comprising the sequence of SEQ ID NO:1 or SEQ ID NO:2 and still binds Neu5Gc-Lewis^{a} specifically because it does not bind any other form of Neu5Gc or any other Sialyl-Lewis^{a}, including free Neu5Gc. In one embodiment, the methods of the disclosure may be practiced with a peptide having at least 90%, 91%, 92%, 93%, 94%, 95%, 96%, 97%, 98%, or 99% homology or identity to a peptide comprising the sequence of SEQ ID NO:1 or SEQ ID NO:2, that specifically binds Neu5Gc-Lewis^{a} specifically because it does not bind any other form of Neu5Gc or any other Sialyl-Lewis^{a}, including free Neu5Gc. In some embodiments, the peptide has at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% homology or identity to a peptide comprising the sequence of SEQ ID NO:1 or SEQ ID NO:2, that specifically binds Neu5Gc-Lewis^{a} specifically because it does not bind any other form of Neu5Gc or any other Sialyl-Lewis^{a}, including free Neu5Gc. In some embodiments, the peptide is truncated and lacks up to 5, 4, 3, 2 or 1 contiguous or non-contiguous amino acids relative to a peptide comprising the sequence of SEQ ID NO:1 or SEQ ID NO:2, that specifically binds Neu5Gc-Lewis^{a} specifically because it does not bind any other form of Neu5Gc or any other Sialyl-Lewis^{a}, including free Neu5Gc. In one embodiment, the methods are practiced with any one of the versions of MGS5.

Only the monomer MGS5 peptide sequences are shown above, however, MGS5 peptides can be used as multimers, such as dimers, trimers and tetramers. In some aspects, the MGS peptides disclosed herein can be chemically conjugated to another MGS peptide, a cargo and/or another linker. In an aspect, the one or more MGS peptides can be chemically conjugated to an agent (e.g., a cargo molecule). In an aspect, the one or more MGS peptides can be conjugated to polyethylene glycol (PEGT). In an aspect, the number of PEG units can be 1, 2, 3, 4, 5, 6, 7, 8, 9, 10, 11, or more. In aspect, the number of PEG units can be of sufficient length to separate the one or more MGS peptides from one or more conjugated agent (e.g., cargo molecule) to prevent any steric interference between the one or more MGS peptides and the conjugated agent.

In some embodiments, MGS5 peptides can be modified. In some embodiments, modifying an MGS5 peptide comprises optimizing the peptide or stabilizing the peptide. Optimized peptides can be obtained by applying modifications to the individual parental peptide sequences. Modification at the amino-terminus by acetylation (CH₃CO—) and/or d-amino acids, such as d(Leu) can protect against degradation by peptidases. Thus, in some embodiments, the MGS5 peptide can have an N-terminal protection group. In some aspects, the N-terminal protection group can be anything that prevents proteases from cleaving the amino acids from the N-terminus. In some aspects, the MGS5 peptides disclosed herein can be modified by acetylation on the N-terminus. In some embodiments, the N-terminal protection group is an acetyl group (Ac═CH₃CO). In some embodiments, the N-terminal protection group can be, but is not limited to, PEG, Formyl, CH₃—(CH)n-CO, Fluorophore, Fatty acid, alkyl amine, aryl groups, carbohydrates, sulfonamide, or carbamate.

The substitution of like amino acids may be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0+1), glutamate (+3.0+1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5+1), alanine (−0.5), and glycine (0); hydrophobic, aromatic amino acids: tryptophan (−3.4), phenylalanine (−2.5), and tyrosine (−2.3). An amino acid may be substituted for another having a similar hydrophilicity and produce a biologically or immunologically modified protein. In such changes, the substitution of amino acids whose hydrophilicity values are within +2 is preferred, those that are within +1 are particularly preferred, and those within +0.5 are even more particularly preferred. Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include arginine and lysine; glutamate and aspartate; serine and threonine; glutamine and asparagine; and valine, leucine and isoleucine.

In one embodiment, a conservative amino acid substitution is one in which an amino acid residue is replaced with an amino acid residue having a similar side chain, for example amino acids with basic side chains (e.g., lysine, arginine, histidine); acidic side chains (e.g., aspartic acid, glutamic acid); uncharged polar side chains e.g., glycine, asparagine, glutamine, serine, threonine, tyrosine, cysteine, histidine); nonpolar side chains (e.g., alanine, valine, leucine, isoleucine, proline, phenylalanine, methionine, tryptophan); beta-branched side chains (e.g., threonine, valine, isoleucine), and aromatic side chains (e.g., tyrosine, phenylalanine, tryptophan). Particular examples include the substitution of one hydrophobic residue, such as isoleucine, valine, leucine or methionine, for another, or the substitution of one polar residue for another, such as the substitution of arginine for lysine, glutamic acid for aspartic acid, or glutamine for asparagine, serine for threonine, and the like. Proline, which is considered more difficult to classify, shares properties with amino acids that have aliphatic side chains (e.g., Leu, Val, Ile, Gly, and Ala). In certain circumstances, substitution of glutamine for glutamic acid or asparagine for aspartic acid may be considered a similar substitution in that glutamine and asparagine are amide derivatives of glutamic acid and aspartic acid, respectively.

One of ordinary skill in the art is familiar with a variety of methods to calculate identity or homology of one peptide to the other. In one embodiment, to calculate percent identity, the sequences being compared are typically aligned in a way that gives the largest match between the sequences. One example of a computer program that may be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two peptides for which the percent sequence identity is to be determined. The sequences are aligned for optimal matching of their respective amino acid (the “matched span,” as determined by the algorithm.) In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm. In some embodiments, identity may be determined as percentage of identity using known computer algorithms such as the “FASTA” program, using for example, the default parameters as in Pearson et al. (1988) Proc. Natl. Acad. Sci. USA 85:2444 (other programs include the GCG program package (Devereux, I, et al., Nucleic Acids Research 12(I):387 (1984)), BLASTP, BLASTN, FASTA Atschul, S. F., et al, J Molec Biol 215:403 (1990); Guide to Huge Computers, Mrtin J. Bishop, ed., Academic Press, San Diego, 1994, and Carillo et al. (1988) SIAM J Applied Math 48: 1073). For example, the BLAST function of the National Center for Biotechnology Information database may be used to determine identity. Other commercially or publicly available programs include “MegAlign” program (Madison, Wis.) and the University of Wisconsin Genetics Computer Group (UWG) “Gap” program (Madison Wis.).

In one embodiment, the Neu5Gc-Lewis^{a}-binding molecule is labeled. In one embodiment, the label facilitates the detection of the binding to Neu5Gc-Lewis^{a}. In some embodiments, the label is a fluorescence label. In some embodiments, the label is biotinylated. In some embodiments, the label is a radiolabel. In some embodiments, the binding takes place in vivo. In some embodiments, the labeled Neu5Gc-Lewis^{a}-binding molecule may be used to image a cancer or diseased tissue to which it binds. In some embodiments, the binding takes place in vitro.

In one embodiment, the Neu5Gc-Lewis^{a}-binding molecule is identified by screening a library of molecules for binding to Neu5Gc-Lewis^{a}. In some embodiments, the molecules are a peptide, antibody, nucleic acid, a carbohydrate, a lipid, a small organic molecule, a lipid particle (e.g., nanoparticle), or an aptamer. In some embodiments, the libraries are combinatorial libraries. In some embodiments, the library is a chemical peptide library. In some embodiments, the library is a biological library. In some embodiments, the libraries are phage libraries. New molecule identification via library production and screening is a technique well described in the art.

In one embodiment, the disclosure provides Neu5Gc-Lewis^{a}-binding molecules that have been chemically or physically modified. In some embodiments, the chemical modification comprises pegylation (directly or through a linker), acetylation, a d-amino acid, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphatidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, proteolytic processing, phosphorylation, prenylation, racemization, selenoylation, sulfation, and/or transfer-RNA mediated addition of amino acids to protein (e.g., arginylation), cysteamide, a cysteine, a thiol, an amide, a nitrilotriacetic acid optionally substituted, a carboxyl, a linear or ramified Ci-Ce alkyl optionally substituted, a primary or secondary amine, an osidic derivative, etc.). Other modifications are available in the art to one of ordinary skill in the art and are within the scope of the disclosure. In one embodiment, Neu5Gc-Lewis^{a}-binding molecules are incorporated into lipid vesicles or nanoparticles, covalently or non-covalently. In some embodiments, the nanoparticle comprises a porous inorganic nanoparticle, a metal-organic framework nanoparticle, or a porous organic nanoparticle. In certain other embodiments, the nanoparticle is a solid nanoparticle and the cargo may be disposed within (e.g., intermixed with) the material forming the nanoparticle or adsorbed to, or covalently or ionically bound to, the nanoparticle surface). In certain embodiments the nanocarrier comprises a lipid bilayer encasing (or surrounding or enveloping) the particle core. In certain embodiments the nanocarrier is a liposome and the cargo may be disposed within the liposome or nanoparticle. In some embodiments, the Neu5Gc-Lewis^{a}-binding molecules are conjugated, directly or through a linker, to a cargo molecule. In some embodiment, the Neu5Gc-Lewis^{a}-binding molecules is covalently linked to the cargo via a cleavable or non-cleavable linker (e.g., bifunctional, trifunctional linker, or multi-functional linker.

In certain embodiments, the Neu5Gc-Lewis^{a}-binding molecule is linked to a cargo molecule or delivery vehicle. In some embodiments, the Neu5Gc-Lewis^{a}-binding molecules binds to a cell surface molecule of a target cell of interest, thereby directing the cargo or delivery vehicle to the target cell.

In one embodiment, the cargo or delivery vehicle is operably linked to a targeting molecule that comprises a targeting domain comprising at least one MGS5 peptide that promotes targeting of the cargo or delivery vehicle to a cancer cell. In some embodiments, the targeting domain comprises at least one MGS5 peptide selected from SEQ ID NO:1 or SEQ ID NO:2, or a fragment or variant thereof. For example, in some embodiments, a dimer of an MGS5 peptide may be conjugated to a PEG moiety on a lipid nanoparticle (LNP) to direct the LNP to a cancer cell. In some embodiments, the LNP encapsulates a therapeutic agent for the treatment of the cancer.

### VII. LINKERS

Disclosed are linkers. In some aspects, the linkers can conjugate or link two or more MGS peptides together, two or more linkers together, or a linker or peptide to a cargo.

In some aspects, a linker that conjugates two or more MGS peptides together can be referred to as a dimeric core (if linked to two peptides) or a tetrameric core (if linked to four peptides). In some aspects, any length of PEG can be used. For example, any of PEG 1-PEG30 can be used. In some aspects, a PEG of length 1-5000 can be used. In some aspects, any linker can be used in place of PEG 12 of the dimeric core or tetrameric core.

Disclosed are linkers comprising at least one reactive group capable of binding a C-terminus of a peptide; and at least one additional reactive group capable of chemically reacting with a moiety.

In some aspects, the linker has a length of up to PEG5000. With regards to PEG linkers, in some aspects, the length of the linker can be a single PEG all the way up to 5000 PEGs. In some aspects, the linker between the peptide and the cargo can be longer than the linker between two MGS peptides. In some aspects, the linker comprises two to four PEG linkers. In some aspects, a linker with two PEG linkers can be referred to as a dimeric core. In some aspects, a linker with four PEG linkers can be referred to as a tetrameric core.

In some aspects, a linker comprises at least two PEG linkers and a reactive group between at least two of the PEG linkers. In some aspects, the reactive group connects at least two PEG linkers.

In some aspects, the linker comprises an amino acid, a peptide, an alkyl group, a maleimide, a thiol, hydrazone, or amide. In some aspects, the amino acid can be a modified amino acid. For example, a modified amino acid can be a functionalized lysine, functionalized cysteine, functionalized glutamic acid, or functionalized aspartic acid. In some aspects, the linker comprises biotin.

In some aspects, the reactive group can be, but is not limited to, carboxylic acid, acyl halides, sulfonyl halides, chloroformates, aldehydes, alkynes, alkynes (with No Acetylenic Hydrogen), amides and imides, amines, phosphines, and pyridines, anhydrides, azo, diazo, azido, hydrazine, and azide compounds, carbamates, epoxides, esters, sulfate esters, phosphate, esters, thiophosphate esters, and borate esters, halogenated organic compounds, isocyanates and isothiocyanates, ketones, oximes, sulfides (Organic).

In some aspects, a moiety can be a cargo. In some aspects, a cargo can be, but is not limited to, a dye, an imaging agent, a therapeutic, a protein, a nucleic acid, an amino acid, a peptide, a lipid, an antibody, a radionuclide, carbohydrate or a nanoparticle. In some aspects, the moiety can be a linker. Therefore, in some aspects, disclosed are linkers comprising linkers. For example, if the linker is a tetrameric core (i.e., first linker), the tetrameric core can comprise moiety that is a second linker. In some aspects, a second linker can comprise the same elements or different elements from the tetrameric core. In some aspects, a second linker conjugates a tetrameric core (i.e., third linker) to a dimeric core.

In some aspects, any of the first, second, or third linkers can comprise an amino acid, a peptide, an alkyl group, a maleimide, a thiol, a hydrazone, dibenzocyclooctyne, azide, or an amide.

### VIII. CELLS

In some embodiments, the disclosure provides cells that have been modified to comprise a Neu5Gc-Lewis^{a} binding molecule. In some embodiments, the molecule is on the cell surface. In some embodiments, the Neu5Gc-Lewis^{a} binding molecule helps direct the cell to another cell or tissue (target cell or tissue) that comprises Neu5Gc-Lewis^{a} on the cell surface and/or ECM. In some embodiments, the target cell is a cancer cell. Non-limiting examples of cancers that may be targeted by the cells of the disclosure have been described elsewhere. In some embodiments, the cells are immune cells, which are then referred to as modified immune cells. In some embodiments, the cells are T cells or natural killer (NK) cells. In some embodiments, the Neu5Gc-Lewis^{a} binding molecule, or a multimer (e.g., dimer) thereof, is part of a chimeric antigen receptor (CAR). For example, in some embodiments, provided are CAR molecules comprising a Neu5Gc-Lewis^{a} binding domain, wherein the Neu5Gc-Lewis^{a} binding domain comprises a dimer of SEQ ID NO:1 or SEQ ID NO:2, linked by a liker molecule. In some embodiments, the cell is a CAR-T cell, a CAR-NK cell or a tumor infiltrating lymphocyte.

The modified immune cells expressing the CARs may be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naïve T lymphocytes or MHC-restricted T lymphocytes. In certain embodiments, the T lymphocytes are tumor infiltrating lymphocytes (TILs). In certain embodiments, the T lymphocytes have been isolated from a tumor biopsy or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR may be isolated using art-accepted, routine methods, e.g., blood collection followed by apheresis and optionally antibody-mediated cell isolation or sorting. The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes are used to prepare modified T lymphocytes, it is preferable to select T lymphocytes that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes may be reduced by co-administration to the host of one or more immunosuppressive agents, e.g., cyclosporine, tacrolimus, sirolimus, cyclophosphamide, or the like.

The disclosure also provides compositions comprising these cells, which compositions may be as described above for the Neu5Gc-Lewis^{a} binding molecules of the disclosure. These cells may be administered by any of the methods described elsewhere in the specification.

Desirably, an effective amount or sufficient number of the isolated modified immune cells is present in the composition and introduced into the subject such that long term, specific, anti-tumor responses are established to reduce the size of a tumor or eliminate tumor growth or regrowth than would otherwise result in the absence of such treatment. Desirably, the amount of T cells reintroduced into the subject causes a 10%, 20%, 30%, 40%, 50%, 60%, 70%, 80%, 90%, 95%, 98%, or 100% decrease in tumor size when compared to otherwise same conditions wherein the T cells are not present. As used herein the term “anti-tumor effective amount” refers to an effective amount of CAR-expressing immune effector cells to reduce cancer cell or tumor growth in a subject. Furthermore, the amounts of each active agent included in the compositions described herein (e.g., the amount per each cell to be contacted or the amount per certain body weight) may vary in different applications. In general, the concentration of T cells desirably should be sufficient to provide in the subject being treated at least from about 1×10⁶ to about 1×10⁹ transduced T cells, even more desirably, from about 1×10⁷ to about 5×10⁸ transduced T cells, although any suitable amount may be utilized either above, e.g., greater than 5×10⁸ cells, or below, e.g., less than 1×10⁷ cells. The dosing schedule may be based on well-established cell-based therapies (see, e.g., U.S. Pat. No. 4,690,915), or an alternate continuous infusion strategy may be employed.

In some embodiments, the Neu5Gc-Lewis^{a}-binding molecule binds to or targets a cell, provided that the cell comprises Neu5Gc-Lewis^{a}. While healthy cells do not appear to comprise Neu5Gc-Lewis^{a}, the disclosure provides that there may be embodiments where a cell does comprise Neu5Gc-Lewis^{a}, in which case it may be targeted by the Neu5Gc-Lewis^{a}-binding molecules of the disclosure. For those embodiments, exemplary cells may include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes, [0245] In some embodiments, the target cell is a cell of a target tissue. The target tissue may include liver, lungs, heart, spleen, pancreas, gastrointestinal tract, kidney, testes, ovaries, brain, reproductive organs, central nervous system, peripheral nervous system, skeletal muscle, endothelium, inner ear, or eye. [0246] In some embodiments, the target cell is a muscle cell (e.g., skeletal muscle cell), kidney cell, liver cell (e.g., hepatocyte), or a cardiac cell (e.g., cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g., a naïve T cell), a macrophage (e.g., a tumor infiltrating macrophage), or a fibroblast (e.g., a cardiac fibroblast). In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, an endothelial cell, or a non-cancerous cell).

### IX. DRUG AND CELL TARGETING

The disclosure also provides methods of targeting diseased cells and tissues and/or delivering a cargo such as, without limitation, therapeutic agents or diagnostic agents to the same. In one embodiment, the disclosure provides a method of targeted delivery of a cargo molecule to a cell known to, or suspected of, express Neu5Gc-Lewis^{a} on its cell surface, comprising contacting the cell with a Neu5Gc-Lewis^{a} binding molecule linked to a cargo, directly or through a linker (e.g., bifunctional, trifunctional linker, or multi-functional linker). In some embodiments, the linker may be a cleavable or non-cleavable linker. In one embodiment, the Neu5Gc-Lewis^{a} binding molecule comprises a peptide, antibody, nucleic acid, a carbohydrate, a lipid, a small organic molecule, a lipid particle (e.g., nanoparticle), or an aptamer. In one embodiment, the Neu5Gc-Lewis^{a} binding molecule binds specifically to Neu5Gc-Lewis^{a}. In one embodiment, the Neu5Gc-Lewis^{a} binding molecule has been previously determined not to bind to any other form of Neu5Gc or any other Sialyl-Lewis^{a}, other than Neu5Gc-Lewis^{a} on the cell, including free Neu5Gc. In one embodiment, the cell is a cancer cell. In one embodiment, the tissue is cancer tissue. In one embodiment, the tissue is diseased tissue which has been identified as comprising areas comprising Neu5Gc-Lewis^{a} on the cell surface and/or the ECM. In one embodiment, the tissue is atherosclerotic tissue. Non-limiting examples of cancers and other tissues are described elsewhere in this disclosure. In some embodiments, the cargo is delivered to the cell surface or extracellular matrix. In some embodiments, the cargo is delivered intracellularly.

The disclosure provides that any cell, exosome or tissue may be targeted by a Neu5Gc-Lewis^{a}-binding molecule, provided that it comprises Neu5Gc-Lewis^{a}. In some embodiments, the Neu5Gc-Lewis^{a} is on the cell surface. In some embodiments, the Neu5Gc-Lewis^{a} is on an exosome. In some embodiments, the Neu5Gc-Lewis^{a} is in the ECM. In some embodiments, the Neu5Gc-Lewis^{a} is in a combination of any of the cell surface, an exosome or in the ECM. In some embodiments, the Neu5Gc-Lewis^{a} is intracellular.

In some embodiments, a cargo molecule is linked to a Neu5Gc-Lewis^{a} binding molecule and is delivered to a cell, exosome or tissue comprising Neu5Gc-Lewis^{a}. In some embodiments, the cargo molecule is delivered extracellularly and/or intracellularly.

In some embodiments, the cargo comprises a labeling agent (e.g., a visualization agent, a diagnostic agent). In some embodiments, the cargo comprises a therapeutic agent. In some embodiments, the cargo comprises a molecule selected from peptides, nucleic acids (e.g., siRNA, antisense oligonucleotides, microRNA, shRNA, miRNA, shRNA, gRNA, mRNA, DNA, DNA plasmid, an oligonucleotide, and an analogue thereof), proteins (including, but not limited to, streptavidin, phycoerythrin, a cytokine, an interleukin, an enzyme, a receptor, a microprotein, a hormone, erythropoietin, a ribonuclease (RNAse), a deoxyribonuclease (DNAse), a blood clotting factor, an anticoagulant, a bone morphogenetic protein, an engineered protein scaffold, a thrombolytic protein, a CRISPR protein, granulocyte-macrophage colony-stimulating factor (GMCSF), a transcription factor, a transposon, a reverse transcriptase, a viral interferon antagonist), fluorophores, liposomes, polysaccharides, nanoparticles, small molecule therapeutics (including, but not limited to, MMAF, DM1, Amanitin, Duocarmycin, doxorubicin, paclitaxel), PET dyes, NIR dyes, MRI agents, antibodies, ribonucleoproteins, antigenic peptides, protein toxins (including, but not limited to, saporin), a genome editing system.

In some embodiments, the linker comprises a polyglycine linker. In some embodiments, the linker is selected from the group consisting of beta alanine, cysteine, cysteamide bridge, poly glycine (such as G2 or G4), a PEG linker moiety, Aun (11-amino-undecanoic acid), Ava (5-amino pentanoic acid), and Ahx (aminocaproic acid). In some embodiments, the linker comprises a PEG linker moiety. In some embodiments, the PEG linker moiety consists of about one to ten (such as about 1-8, 2-7, 1-5, or 6-10) ethylene glycol units. In some embodiments, the molecular weight of the PEG linker moiety is about 0.05 kDa to about 0.5 kDa (such as about 0.05-0.1, 0.05-0.4, 0.1-0.3. 0.05-0.25, 0.25-0.5 kDa). In some embodiments, the PEG linker moiety is a linear PEG. In some embodiments, the PEG linker moiety is a branched PEG. In some embodiments, the linker comprises a beta-alanine. In some embodiments, the linker comprises at least about two, three, or four glycines, optionally continuous glycines. In some embodiments, the linker further comprises a serine. In some embodiments, the linker comprises a GGGGS (SEQ ID NO:3) or SGGGG (SEQ ID NO:4) sequence. In some embodiments, the linker comprises a Glycine-b-Alanine motif. In some embodiments, the cargo may be attached to the Neu5Gc-Lewis^{a}-binding molecule at an amino group, carboxylate group, or the side chain of any of the amino acids of the Neu5Gc-Lewis^{a}-binding molecule (e.g., at the amino group, the carboxylate group, or the side chain.

In one embodiment of the disclosure, a Neu5Gc-Lewis^{a}-binding molecule (e.g., a peptide) is linked with a drug via a bifunctional crosslinking reagent. As used herein, a “bifunctional crosslinking reagent” refers to a reagent that possesses two reactive groups one of which is capable of reacting with a Neu5Gc-Lewis^{a}-binding molecule, while the other one is capable of reacting with the drug to link the Neu5Gc-Lewis^{a}-binding molecule with the drug, thereby forming a conjugate. Any suitable bifunctional crosslinking reagent may be used in connection with the disclosure, so long as the linker reagent provides for retention of the drug, e.g., cytotoxicity, and targeting characteristics of the Neu5Gc-Lewis^{a}-binding molecule. Preferably, the linker molecule joins the drug to the Neu5Gc-Lewis^{a}-binding molecule through chemical bonds, such that the drug and the Neu5Gc-Lewis^{a}-binding molecule are chemically coupled (e.g., covalently bonded) to each other.

In one embodiment, the bifunctional crosslinking reagent comprises non-cleavable linkers. A non-cleavable linker is any chemical moiety that is capable of linking a drug to a Neu5Gc-Lewis^{a}-binding molecule in a stable, covalent manner. Preferably, a non-cleavable linker is not cleavable under physiological conditions, in particular inside the body and/or inside a cell. Thus, non-cleavable linkers are substantially resistant to acid-induced cleavage, light-induced cleavage, peptidase-induced cleavage, esterase-induced cleavage, and disulfide bond cleavage, at conditions under which the drug or the Neu5Gc-Lewis^{a}-binding molecule remains active. Suitable crosslinking reagents that form non-cleavable linkers between a drug and a Neu5Gc-Lewis^{a}-binding molecule are well known in the art. In one embodiment, the drug is linked to the Neu5Gc-Lewis^{a}-binding molecule through a thioether bond.

In one particularly preferred embodiment, the linking reagent is a cleavable linker. Preferably, a cleavable linker is cleavable under physiological conditions, in particular inside the body and/or inside a cell. Examples of suitable cleavable linkers include disulfide linkers, acid labile linkers, photolabile linkers, peptidase labile linkers, and esterase labile linkers.

Non-limiting examples of linkers include, but are not limited to, N-succinimidyl-3-(2-pyridyldithio)butyrate (SPDB), N-succinimidyl-3-(2-pyridyldithio)propionate (SPDP), sulfosuccinimidyl-4-(N-maleimidomethyl)cyclohexane-1-carboxylate (Sulfo-SMCC), N-succinimidyl-4-(maleimidomethyl)cyclohexanecarboxylate (SMCC), N-succinimidyl-4-(N-maleimidomethyl)-cyclohexane-1-carboxy-(6-amidocaproate) (LC-SMCC), 4-maleimidobutyric acid N-hydroxysuccinimide ester (GMBS), 3-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl-4-(p-maleimidophenyl)-butyrate (SMPB), N-(p-maleimidophenypisocymate (PMPI), 6-maleimidocaproyl (MC), maleimidopropanoyl (MP), p-aminobenzyloxycarbonyl (PAB), N-succinimidyl-4-(2-pyridylthio)pentanoate (SPP), and N-succinimidyl (4-iodoacetyl)aminobenzoate (SIAB). A peptide linker such as valine-citrulline (Val-Cit) or alanine-phenylalanine (ala-phe) may also be used, and any of the aforementioned linkers may be used in adequate combination.

Disulfide containing linkers are linkers cleavable through disulfide exchange, which may occur under physiological conditions. In yet other embodiments, the linker is cleavable under reducing conditions (e.g., a disulfide linker). A variety of disulfide linkers are known in the art, including, for example, those that may be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)

Acid labile linkers are linkers cleavable at acidic pH. For example, certain intracellular compartments, such as endosomes and lysosomes, have an acidic pH (pH 4-5), and provide conditions suitable to cleave acid labile linkers. Acid labile linkers are relatively stable under neutral pH conditions, such as those in the blood, but are unstable at below pH 5.5 or 5.0. For example, a hydrazone, semicarbazone, thiosemicarbazone, cis-aconitic amide, orthoester, acetal, ketal, or the like may be used. Photolabile linkers are useful at the body surface and in many body cavities that are accessible to light. Furthermore, infrared light may penetrate tissue. Peptidase labile linkers may be used to cleave certain peptides inside or outside cells. In one embodiment, the cleavable linker is cleaved under mild conditions, i.e., conditions within a cell under which the activity of the cytotoxic agent is not affected.

The linker may be or may comprise, e.g., a peptidyl linker that is cleaved by an intracellular peptidase or protease enzyme, including, but not limited to, a lysosomal or endosomal protease. Typically, the peptidyl linker is at least two amino acids long or at least three amino acids long. Cleaving agents may include cathepsins B and D and plasmin, all of which are known to hydrolyze dipeptide drug derivatives resulting in the release of active drug inside target cells. For example, a peptidyl linker that is cleavable by the thiol-dependent protease cathepsin-B, which is highly expressed in cancerous tissue, may be used (e.g., a Phe-Leu or a Gly-Phe-Leu-Gly linker). In specific embodiments, the peptidyl linker cleavable by an intracellular protease is a valine-citrulline (Val-Cit; vc) linker or a phenylalanine-lysine (Phe-Lys) linker. One advantage of using intracellular proteolytic release of the therapeutic agent is that the agent is typically attenuated when conjugated and the serum stabilities of the conjugates are typically high.

### X. NON-LIMITING EXAMPLES OF THERAPEUTIC AGENTS

In one embodiment, the therapeutic agent comprises an agent that works as an anti-cancer agent. In some embodiments, the therapeutic agent comprises a cell of the disclosure, a therapeutic antibody or a chemotherapeutic agent. In one embodiment, the therapeutic agent is selected from the group consisting of an antibody or fragment thereof, a toxin, a radionuclide, an immunomodulator, a radiosensitizing agent, a hormone, an anti-angiogenesis agent, and combinations thereof. In other embodiments, the other therapeutic agent is an anti-tumor antibiotic (e.g., actinomycin D, doxorubicin, daunorubicin, Epirubicin, mitomycin, pelomycin, pingyangmycin, pirarubicin), antiviral, anti-inflammatory agent, a cytokine, a hematopoietic growth factor, anti-cancer agent (including a chemotherapeutic agent), an immunomodulatory agent, an immunosuppressive agent, a steroid (e.g., a corticosteroid) or a pharmacologically active derivative thereof, a vitamin, calcium or a calcium supplement, and the like.

In one embodiment, the anti-cancer agent is a therapeutic antibody. In one embodiment, the antibody or functional fragment thereof is selected from the group consisting of anti-Her2 antibody, anti-EGFR antibody, anti-VEGFR antibody, anti-CD20 antibody, anti-CD33 antibody, anti-PD-L1 antibody, anti-PD-1 antibody, anti-CTLA-4 antibody, anti-TNFα antibody, anti-CD28 antibody, anti-4-1BB antibody, anti-OX40 antibody, anti-GITR antibody, anti-CD27 antibody, anti-CD40 antibody, or anti-ICOS antibody, anti-CD25 antibody, anti-CD30 antibody, anti-CD3 antibody, anti-CD22 antibody, anti-CCR4 antibody, anti-CD38 antibody, anti-CD52 antibody, anti-Complement C5 antibody, anti-F protein of RSV, anti-GD2 antibody, anti-GITR antibody, anti-Glycoprotein receptor lib/Illa antibody, anti-ICOS antibody, anti-IL2R antibody, anti-LAG3 antibody, anti-Integrinα4 antibody, anti-lgE antibody, anti-PDGFRα antibody, anti-RANKL antibody, anti-SLAMF7 antibody, anti-LTIGIT antibody, anti-TIM-3 antibody, anti-VEGFR2 antibody, anti-VISTA antibody, Utomilumab, Urelumab, ADG106, Poteligeo™ (Mogamulizumab), Poteligeo™ (Mogamulizumab), Bexxar™ (tositumomab), Zevalin™ (ibritumomab tiuxetan), Rituxan™ (rituximab), Arzerra™ (Ofatumumab), Gazyva™ (Obinutuzumab), Besponsa™ (Inotuzumab ozogamicin), Zenapax™ (daclizumab), Varlilumab, Theralizumab, Adcetris™ (Brentuximab vedotin), Myelotarg™ (gemtuzumab), Darzalex™ (Daratumumab), CDX-1140, SEA-CD40, R07009789, JNJ-64457107, APX-005M, Chi Lob 7/4, Campath™ (alemtuzumab), Raptiva™ (efalizumab), Soliris™ (eculizumab), Yervoy™ (ipilimumab), tremelimumab, Erbitux™ (cetuximab), Vectibix™ (panitumumab), Portrazza™ (Necitumumab), TheraCIM™ (Nimotuzumab), Synagis™ (palivizumab), Unituxin™ (Dinutuximab), TRX-518, MK-4166, MK-1248, GWN-323, INCAGN0186, BMS-986156, AMG-228, ReoPro™ (abiciximab), Herceptin™ (trastuzumab), Perjeta™ (Pertuzumab), Kadcyla™ (Ado-trastuzumab emtansine), GSK-3359609, JTX-2011, Simulect™ (basiliximab), Tysabri™ (natalizumab), BMS-986016, REGN3767, LAG525, Xolair™ (omalizumab), Tavolimab, PF-04518600, BMS-986178, MOXR-0916, GSK-3174998, INCAGN01949, IBI-101, Keytruda™ (Pembrolizumab), Opdivo™ (Nivolumab), Lartruvo™ (Olaratumab), Tencentriq™ (Atezolizumab), BMS-936559, Bavencio™ (Avelumab), Imfinzi™ (Duralumab), Prolia™ (Denosumab), Empliciti™ (Elotuzumab), MTIG7192A, TSR-022, MBG-453, Remicade™ (infliximab), Humira™ (adalimumab), Avastin™ (bevacizumab), Lucentis™ (ranibizumab), Cyramza™ (Ramucirumab), and JNJ-61610588.

In one embodiment, the therapeutic agent is a “check point inhibitor.” The term “check-point inhibitor” refers to molecules that totally or partially reduce, inhibit, interfere with or modulate one or more checkpoint proteins. Without being limited by a particular theory, checkpoint proteins regulate T cell activation or function. Numerous checkpoint proteins are known, such as CTLA-4 and its ligands CD80 and CD86; and PD-1 with its ligands PD-L1 and PD-L2. These proteins appear responsible for co-stimulatory or inhibitory interactions of T cell responses. Immune check-point proteins appear to regulate and maintain self-tolerance and the duration and amplitude of physiological immune responses.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammal1 and calicheamicin omegal1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholinodoxorubicin, cyanomorpholino-doxorubicin, 2-pyrrolino-doxorubicin and deoxy doxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DFMO); retinoids, such as retinoic acid; capecitabine; carboplatin, procarbazine, plicomycin, gemcitabien, navelbine, farnesyl-protein transferase inhibitors, transplatinum, 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, LCR, and pharmaceutically acceptable salts, acids, or derivatives of any of the above.

Many tumors and cancers have viral genome present in the tumor or cancer cells. For example, Epstein-Barr Virus (EBV) is associated with a number of mammalian malignancies. The compounds disclosed herein may also be used alone or in combination with anti-cancer or antiviral agents, such as ganciclovir, azidothymidine (AZT), lamivudine (3TC), etc., to treat patients infected with a virus that may cause cellular transformation and/or to treat patients having a tumor or cancer that is associated with the presence of viral genome in the cells. The compounds disclosed herein may also be used in combination with viral based treatments of oncologic disease.

### XI. COMPOSITIONS

Provided herein are compositions/therapeutic formulations comprising Neu5Gc-Lewis^{a}-binding molecule and another component, such as a carrier. Also provided are pharmaceutical compositions/therapeutic formulations comprising a Neu5Gc-Lewis^{a}-binding molecule and an excipient and/or diluent. In one embodiment, the carrier is not a naturally existing compound. In one embodiment, the excipient is not a naturally existing compound. In another embodiment, the diluent is not a naturally existing compound. In another embodiment, the formulation comprising the Neu5Gc-Lewis^{a}-binding molecule does not contain a naturally existing compound, except, optionally, water. It will be apparent to those persons skilled in the art that certain carriers, excipients, or diluents may be more preferably depending upon, for instance, the route of administration and concentration of Neu5Gc-Lewis^{a}-binding molecule being administered. In all embodiments, including molecules and compositions for administration, any references to “Neu5Gc-Lewis^{a}-binding molecule” include the “naked” molecule as well as modifications to the “naked” molecule (i.e., modified Neu5Gc-Lewis^{a}-binding molecule), including, for example, addition (direct or indirect linkage or conjugation) of a cargo molecule (e.g., a therapeutic agent, a label, etc.), incorporation into a lipid vesicle, chemical modifications, any other modifications to the “naked” molecule. In one embodiment, the “naked” Neu5Gc-Lewis^{a}-binding molecule is a peptide or an antibody and the peptide or antibody is chemically modified (e.g., acylation, pegylation, etc.) and conjugated, directly or through a linker, to a cargo molecule. Non-limiting examples of modifications and cargo molecules are described elsewhere in the application.

In one embodiment, the disclosure provides pharmaceutical compositions of Neu5Gc-Lewis^{a}-binding molecule to be used in accordance with the present disclosure. In some embodiments, the compositions are prepared for storage and/or administration by mixing a Neu5Gc-Lewis^{a}-binding molecule having the desired degree of purity with optional pharmaceutically acceptable carriers, diluents, excipients, or stabilizers (Remington's Pharmaceutical Sciences 23rd edition, Adejare, A., Ed. (2020)), in the form of lyophilized formulations or aqueous solutions. In one embodiment, acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN, PLURONICS or polyethylene glycol (PEG). Examples of lyophilized antibody formulations are described in WO 97/04801, expressly incorporated herein by reference.

In a further embodiment, the formulation further comprises a surfactant. The surfactant may, for example, be selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g., Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g., phosphatidyl serine, phosphatidyl choline, phosphatidyl ethanolamine, phosphatidyl inositol, diphosphatidyl glycerol and sphingomyelin), derivates of phospholipids (e.g., dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g., cephalins), glyceroglycolipids (e.g., galactopyransoide), sphingoglycolipids (e.g., ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (e.g., oleic acid and caprylic acid), acylcarnitines and derivatives, N alpha.-acylated derivatives of lysine, arginine or histidine, or side-chain acylated derivatives of lysine or arginine, Nalpha.-acylated derivatives of dipeptides comprising any combination of lysine, arginine or histidine and a neutral or acidic amino acid, Nalpha-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (e.g., Dodecyl .beta.-D-glucopyranoside), poloxamines (e.g., Tetronic's), which are tetrafunctional block copolymers derived from sequential addition of propylene oxide and ethylene oxide to ethylenediamine, or the surfactant may be selected from the group of imidazoline derivatives, or mixtures thereof. In one embodiment, the surfactant is not a naturally existing compound. Each one of these specific surfactants constitutes an alternative embodiment of the disclosure.

One embodiment provides for stable formulations of Neu5Gc-Lewis^{a}-binding molecules, which comprise preferably a phosphate buffer with saline or a chosen salt, as well as preserved solutions and formulations containing a preservative, as well as multi-use preserved formulations suitable for pharmaceutical or veterinary use, comprising at least one the antibodies and/or target-binding fragments thereof in a pharmaceutically acceptable formulation. In one embodiment, preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture may be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like. In one embodiment, the preservative or preservatives are not naturally existing compounds.

In one embodiment, the pharmaceutical composition comprises a Neu5Gc-Lewis^{a}-binding molecule of the disclosure and a pharmaceutically acceptable carrier. In one embodiment, “pharmaceutically acceptable carrier” includes any and all solvents, dispersion media, coatings, antibacterial and antifungal agents, isotonic and absorption delaying agents, and the like that are physiologically compatible. Additional examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the Neu5Gc-Lewis^{a}-binding molecule.

In one embodiment, an appropriate amount of a pharmaceutically-acceptable salt is used in the formulation to render the formulation isotonic. Examples of the carrier include saline, Ringer's solution and dextrose solution. In one embodiment, the pH of the solution is from about 5 to about 8. In another embodiment, the pH is from about 7 to about 7.5. Further carriers include sustained release preparations such as semipermeable matrices of solid hydrophobic polymers containing the Neu5Gc-Lewis^{a}-binding molecule, which matrices are in the form of shaped articles, e.g., films, liposomes or microparticles. A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The compositions of this disclosure may be in a variety of forms. These include, for example, liquid, semi-solid and solid dosage forms, such as liquid solutions (e.g., injectable and infusible solutions), dispersions or suspensions, tablets, pills, powders, liposomes and suppositories. In some embodiments, such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present disclosure may be formulated as a lyophilizate. Neu5Gc-Lewis^{a}-binding molecule may also be used in liposomes using well-known technologies.

Dosage forms suitable for internal administration generally contain from about 0.1 milligram to about 500 milligrams of Neu5Gc-Lewis^{a}-binding molecule (the active ingredient) per unit or container. In these pharmaceutical compositions, the active ingredient will ordinarily be present in an amount of about 0.5-99.999% by weight based on the total weight of the composition.

Therapeutic compositions/formulations typically must be sterile and stable under the conditions of manufacture and storage. The composition may be formulated as a solution, microemulsion, dispersion, liposome, or other ordered structure suitable to high drug concentration. Sterile injectable solutions may be prepared by incorporating the active compound (i.e., Neu5Gc-Lewis^{a}-binding molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution may be maintained, for example, by the use of a coating such as lecithin, by the maintenance of the required particle size in the case of dispersion and by the use of surfactants. Prolonged absorption of injectable compositions may be brought about by including in the composition an agent that delays absorption, for example, monostearate salts and gelatin.

The preferred dosage form depends on the intended mode of administration and therapeutic application. One of ordinary skill in the art is familiar with the procedures for determining such dosages. Typical compositions are in the form of injectable or infusible solutions, such as compositions similar to those used for passive immunization of humans with antibodies. The most typical mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The Neu5Gc-Lewis^{a}-binding molecules of the disclosure may be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, a Neu5Gc-Lewis^{a}-binding molecule may be prepared with a carrier that will protect the compound against rapid release, such as a controlled release formulation, including implants, transdermal patches, and microencapsulated delivery systems. Biodegradable, biocompatible polymers may be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In certain embodiments, a Neu5Gc-Lewis^{a}-binding molecule may be orally administered, for example, with an inert diluent or an assimilable edible carrier. The Neu5Gc-Lewis^{a}-binding molecule may also be enclosed in a hard- or soft-shell gelatin capsule, compressed into tablets, or incorporated directly into the subject's diet. For oral therapeutic administration, a Neu5Gc-Lewis^{a}-binding molecule may be incorporated with excipients and used in the form of ingestible tablets, buccal tablets, troches, capsules, elixirs, suspensions, syrups, wafers, and the like. To administer a Neu5Gc-Lewis^{a}-binding molecule by other than parenteral administration, it may be necessary to coat the Neu5Gc-Lewis^{a}-binding molecule with or co-administer the Neu5Gc-Lewis^{a}-binding molecule with, a material to prevent its inactivation.

In some embodiments, the disclosed compositions are bioavailable and may be delivered orally. Oral compositions may be tablets, troches, pills, capsules, and the like, and may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

In some embodiments, the compositions suitable for injection or infusion may include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity may be maintained, for example, by the formation of liposomes, by the maintenance of the required particle size in the case of dispersions or by the use of surfactants. Optionally, the prevention of the action of microorganisms may be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions may be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein may be applied in as a liquid or solid. However, it will generally be desirable to administer them topically to the skin as compositions, in combination with a dermatologically acceptable carrier, which may be a solid or a liquid. Compounds and agents and compositions disclosed herein may be applied topically to a subject's skin to reduce the size (and may include complete removal) of malignant or benign growths, or to treat an infection site. Compounds and agents disclosed herein may be applied directly to the growth or infection site. Preferably, the compounds and agents are applied to the growth or infection site in a formulation such as an ointment, cream, lotion, solution, tincture, or the like.

Useful dosages of the compounds and agents and pharmaceutical compositions disclosed herein may be determined by comparing their in vitro activity, and in vivo activity in animal models. Methods for the extrapolation of effective dosages in mice, and other animals, to humans are known to the art. The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and may be determined by one of skill in the art. The dosage may be adjusted by the individual physician in the event of any counterindications. Dosage may vary, and may be administered in one or more dose administrations daily, for one or several days. In one embodiment, doses of a peptide of from 1 ng to 1 mg, preferably from 10 ng to 100 μg, are formulated and administered. In one embodiment, if the administration of nucleic acids (DNA and RNA) is desired, doses of from 1 ng to 0.1 mg may be formulated and administered.

In certain embodiments, the therapeutically effective amount of Neu5Gc-Lewis^{a}-binding molecule may be administered in an amount as a measure with regards to the weight of the patient in need thereof. In one embodiment, the Neu5Gc-Lewis^{a}-binding molecule may be administered in an amount of about: 0.1 mg/kg to about 50 mg/kg, 0.1 mg/kg to about 40 mg/kg, 0.1 mg/kg to about 30 mg/kg, 0.1 mg/kg to about 25 mg/kg, 0.1 mg/kg to about 20 mg/kg, 0.1 mg/kg to about 15 mg/kg, 0.1 mg/kg to about 10 mg/kg, 0.1 mg/kg to about 7.5 mg/kg, 0.1 mg/kg to about 5 mg/kg, 0.1 mg/kg to about 2.5 mg/kg, or about 0.1 mg/kg to about 1 mg/kg. The Neu5Gc-Lewis^{a}-binding molecule may be administered in an amount of about: 0.5 mg/kg to about 50 mg/kg, 0.5 mg/kg to about 40 mg/kg, 0.5 mg/kg to about 30 mg/kg, 0.5 mg/kg to about 25 mg/kg, 0.5 mg/kg to about 20 mg/kg, 0.5 mg/kg to about 15 mg/kg, 0.5 mg/kg to about 10 mg/kg, 0.5 mg/kg to about 7.5 mg/kg, 0.5 mg/kg to about 5 mg/kg, 0.5 mg/kg to about 2.5 mg/kg, or about 0.5 mg/kg to about 1 mg/kg. The Neu5Gc-Lewis^{a}-binding molecule may be administered in an amount of about 0.5 mg/kg to about 5 mg/kg or about 0.1 mg/kg to about 10 mg/kg. The Neu5Gc-Lewis^{a}-binding molecule may be administered in an amount of about 0.1 mg/kg to about 20 mg/kg or about 0.1 mg/kg to about 30 mg/kg.

The Neu5Gc-Lewis^{a}-binding molecule may be administered at an amount of about: 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, or 200 mg. The Neu5Gc-Lewis^{a}-binding molecule may be administered at an amount of about: 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The Neu5Gc-Lewis^{a}-binding molecule may be administered at an amount of about 1000 mg to about 2000 mg. The Neu5Gc-Lewis^{a}-binding molecule may be administered at an amount of about: 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 to about 2000 mg.

The Neu5Gc-Lewis^{a}-binding molecule may be administered in an amount of about 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In one embodiment, the Neu5Gc-Lewis^{a}-binding molecule is present in the combination in an amount of about: 1 mg/mL to about 10 mg/mL, 5 mg/mL to about 10 mg/mL, 5 mg/mL to about 15 mg/mL, 10 mg/mL to about 25 mg/mL; 20 mg/mL to about 30 mg/mL, 25 mg/mL to about 50 mg/mL, or 50 mg/mL to about 100 mg/mL.

The Neu5Gc-Lewis^{a}-binding molecule may be administered, for example, once a day (QD), twice daily (BID), once a week (QW), twice weekly (BIW), three times a week (TIW), or monthly (QM) regularly on a continuous base or intermittent base such as BIW for 3 months then resume a month later. In one embodiment, the Neu5Gc-Lewis^{a}-binding molecule may be administered BID. The Neu5Gc-Lewis^{a}-binding molecule may be administered TIW. In certain instances, the Neu5Gc-Lewis^{a}-binding molecule is administered 2 to 3 times a week. In another embodiment, the Neu5Gc-Lewis^{a}-binding molecule is administered QD. The Neu5Gc-Lewis^{a}-binding molecule may be administered QD for about: 1 day to about 7 days, 1 day to about 14 days, 1 day to about 21 days, 1 day to about 28 days, or daily until disease progression or unacceptable toxicity. The administration of Neu5Gc-Lewis^{a}-binding molecule may, in part, depend upon the tolerance of the patient where greater tolerance may allow greater or more frequent administration. Alternatively, where a patient shows poor tolerance to Neu5Gc-Lewis^{a}-binding molecule, a less amount of the compound or a less frequent dosing may be performed. Neu5Gc-Lewis^{a}-binding molecules may be administered in any regimen as described herein.

In one embodiment, a Neu5Gc-Lewis^{a}-binding molecule may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QD. In one embodiment, a Neu5Gc-Lewis^{a}-binding molecule may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, BIW. In one embodiment, a Neu5Gc-Lewis^{a}-binding molecule may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, TIW. In one embodiment, a Neu5Gc-Lewis^{a}-binding molecule may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QW. In one embodiment, a Neu5Gc-Lewis^{a}-binding molecule may be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, Q2W. In one embodiment, a Neu5Gc-Lewis^{a}-binding molecule may be administered at an amount of about 5 mg or about 10 mg, QD. In one embodiment, a Neu5Gc-Lewis^{a}-binding molecule may be administered at an amount of about 5 mg or about 10 mg, BIW. In one embodiment, a Neu5Gc-Lewis^{a}-binding molecule may be administered at an amount of about 5 mg or about 10 mg, TIW. In one embodiment, a Neu5Gc-Lewis^{a}-binding molecule may be administered at an amount of about 5 mg or about 10 mg, QW. In one embodiment, a Neu5Gc-Lewis^{a}-binding molecule may be administered at an amount of about 5 mg or about 10 mg, Q2W. Administration of a Neu5Gc-Lewis^{a}-binding molecule may be continuous. Administration of a Neu5Gc-Lewis^{a}-binding molecule may be intermittent.

In one embodiment, a Neu5Gc-Lewis^{a}-binding molecule may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QD. In one embodiment, a Neu5Gc-Lewis^{a}-binding molecule may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, BIW. In one embodiment, a Neu5Gc-Lewis^{a}-binding molecule may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, TIW. In one embodiment, a Neu5Gc-Lewis^{a}-binding molecule may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QW. In one embodiment, a Neu5Gc-Lewis^{a}-binding molecule may be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, Q2W. Administration of a Neu5Gc-Lewis^{a}-binding molecule may be continuous. Administration of a Neu5Gc-Lewis^{a}-binding molecule may be intermittent.

In one embodiment, a Neu5Gc-Lewis^{a}-binding molecule may be administered at an amount of about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QD. In one embodiment, a Neu5Gc-Lewis^{a}-binding molecule may be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, BIW. In one embodiment, a Neu5Gc-Lewis^{a}-binding molecule may be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, TIW. In one embodiment, a Neu5Gc-Lewis^{a}-binding molecule may be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QW. In one embodiment, a Neu5Gc-Lewis^{a}-binding molecule may be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, Q2W. In one example, a Neu5Gc-Lewis^{a}-binding molecule may be administered at an amount of about 15 mg/kg to about 75 mg/kg, QD. In another example, a Neu5Gc-Lewis^{a}-binding molecule may be administered at an amount of about 20 mg/kg to about 50 mg/kg. In still another example, a Neu5Gc-Lewis^{a}-binding molecule may be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a Neu5Gc-Lewis^{a}-binding molecule may be continuous. Administration of a Neu5Gc-Lewis^{a}-binding molecule may be intermittent.

In one embodiment, a Neu5Gc-Lewis^{a}-binding molecule may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QD. In one embodiment, a Neu5Gc-Lewis^{a}-binding molecule may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, BIW. In one embodiment, a Neu5Gc-Lewis^{a}-binding molecule may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, TIW. In one embodiment, a Neu5Gc-Lewis^{a}-binding molecule may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QW. In one embodiment, a Neu5Gc-Lewis^{a}-binding molecule may be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, Q2W. In one example, a Neu5Gc-Lewis^{a}-binding molecule may be administered at an amount of about 15 mg/kg to about 75 mg/kg, QD. In another example, a Neu5Gc-Lewis^{a}-binding molecule may be administered at an amount of about 20 mg/kg to about 50 mg/kg. In still another example, a Neu5Gc-Lewis^{a}-binding molecule may be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a Neu5Gc-Lewis^{a}-binding molecule may be continuous. Administration of a Neu5Gc-Lewis^{a}-binding molecule may be intermittent.

The Neu5Gc-Lewis^{a}-binding molecule, or any one of the combinations described herein, may be administered in a regimen. The regimen may be structured to provide therapeutically effective amounts of Neu5Gc-Lewis^{a}-binding molecule, or any one of the combinations described herein, over a predetermined period of time (e.g., an administration time). The regimen may be structured to limit or prevent side-effects or undesired complications of each of the components of Neu5Gc-Lewis^{a}-binding molecule, or any one of the combinations described herein described herein. The regimen may be structured in a manner that results in increased effect for both therapies of the combination (e.g., synergy). Regimens useful for treating cancer may include any number of days of administration which may be repeated as necessary. Administration periods may be broken by a rest period that includes no administration of at least one therapy. For example, a regimen may include administration periods that include 2, 3, 5, 7, 10, 15, 21, 28, or more days. These periods may be repeated. For example, a regimen may include a set number of days as previously described where the regimen is repeated 1, 2, 3, 4, 5, 6, 7, 8, 9, 10, 11, 12, 13, or more times.

In other embodiments, the regimens may include a rest period of at least 1, 2, 3, 5, 7, 10, or more days, where at least one therapy is no longer administered to a patient. The rest period may be determined by, for example, monitoring the reaction of the patient to Neu5Gc-Lewis^{a}-binding molecule, or any one of the combinations described herein or by measuring the efficacy of the treatment. A rest period may be applicable to a single therapy, such that only one therapy of a combination described herein is discontinued in the rest period, but the other therapy(ies) are still administered. Rest periods may be applied to all the therapies administered to the subject such that the subject receives no therapy for a set period of time during the rest period.

### XII. COMBINATION TREATMENTS

In some embodiments, the Neu5Gc-Lewis^{a}-binding molecule may be formulated with or conjugated to another therapeutic agent. This agent may be anything that may also be used in the indication for which the Neu5Gc-Lewis^{a}-binding molecule is designed to work.

In some embodiments, the Neu5Gc-Lewis^{a}-binding molecule may be co-administered in combination with another therapeutic agent or treatment (e.g., radiation). In some embodiments, the therapeutic agent or treatment are administered before, concurrently, and/or after the administration of the Neu5Gc-Lewis^{a}-binding molecule. “Co-administered,” as used herein, means that two (or more) different treatments (e.g., a Neu5Gc-Lewis^{a}-binding molecule and an antibiotic) are delivered to the subject during the course of the subject's affliction with the disease, e.g., the two or more treatments are delivered after the subject has been diagnosed with a disease and before the disease has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom (e.g., toxicity resulting from by administration of Neu5Gc-Lewis^{a}-binding molecule), or other parameter related to the disorder is greater than what would be observed with one treatment delivered in the absence of the other. The effect of the two treatments may be partially additive, wholly additive, or greater than additive. The delivery may be such that the effect of the first treatment delivered is still detectable when the second is delivered. The molecules of the disclosure and the additional therapeutic agent may be administered simultaneously, in the same or in separate compositions, or sequentially. In one embodiment, for sequential administration, the Neu5Gc-Lewis^{a}-binding molecule or other molecules described herein may be administered first, and the therapeutic agent may be administered second, or the order of administration may be reversed. The Neu5Gc-Lewis^{a}-binding molecule or any other molecule described herein may be administered during periods of active disorder, or during a period of remission or less active disease. The Neu5Gc-Lewis^{a}-binding molecule or any other molecule described herein may be administered before another treatment, concurrently with the treatment, post-treatment, or during remission of the disorder (e.g., cancer).

### XIII. KITS

In one embodiment, the disclosure provides a kit for the diagnosis or prognosis of cancer and other diseases associated with the presence of Neu5Gc-Lewis^{a} as a biomarker of the same. In some embodiments, the kit comprises a reference sample known to comprise Neu5Gc-Lewis^{a} and means for detecting presence and level of Neu5Gc-Lewis^{a} in a test sample. In one embodiment, the means comprises an anti-Neu5Gc-Lewis^{a} antibody or peptide. In one embodiment, the means for detecting Neu5Gc-Lewis^{a} comprises MGS5 or a peptide having at least 90%, 91%, 92%, 93%, 94%, 95%, 96%, 97%, 98%, or 99% homology or identity to a peptide comprising or consisting of the sequence of SEQ ID NO:1 or SEQ ID NO:2. In some embodiments, a kit of the embodiments comprises a desialylating reagent such as a sialidase, a neuraminidase enzyme, or a mild acid, such as H₂SO₄. Further reagents that may be included in a kit of the embodiments include, without limitation, a microtiter plate, a detectable label, a dilution buffer (e.g., PBS or water), a solid phase carrier, a blocking solution, a chromogenic reagent, a calibrator, and a washing buffer. The blocking solution may be one or more of BSA, bovine serum, skim milk, TBST and other components. The chromogenic solution may be determined according to the labeled substance on the Neu5Gc-Lewis^{a}-binding molecule (e.g., peptide, antibody, etc.), for example, when the labeled substance is horseradish peroxidase, the chromogenic reagent may be luminol.

In some embodiments, the presence of Neu5Gc-Lewis^{a} in the sample is quantitated (for example, for monitoring disease progression or treatment effectiveness). In one embodiment, the quantitative assay is an ELISA assay, a radioimmunoassay (RIA), an immunoradiometric assay, a fluoroimmunoassay, a chemiluminescent immunoassay, a bioluminescent immunoassay, an enzyme multiplied immunoassay (EMIT), a cloned enzyme donor immunoassay (CEDIA), an immuno-PCR assay, a phosphor immunoassay, a quantum dot immunoassay, a solid phase light-scattering immunoassay, a surface effect immunoassay, or an immunoassay employing lateral flow test strips.

In one embodiment, the specificity of the detection of the Neu5Gc-Lewis^{a} biomarker may be confirmed by desialylating the sample with, for example, a sialidase. In one embodiment, the kit may further comprise a standard or control information so that the test sample may be compared with the control information standard to determine if the test amount of Neu5Gc-Lewis^{a} detected in a sample is a diagnostic amount consistent with a diagnosis of a disease, condition or disorder, such as cancer, preferably pancreatic or breast cancer.

The immunodetection methods of the present disclosure have evident utility in the diagnosis and prognosis of conditions such as cancer wherein Neu5Gc-Lewis^{a} is expressed, and wherein antibodies or other Neu5Gc-Lewis^{a}-binding molecules exist that react (immunologically) with Neu5Gc-Lewis^{a}. In one embodiment, a biological and/or clinical sample suspected of containing a specific disease associated with Neu5Gc-Lewis^{a} is used. However, these embodiments also have applications to non-clinical samples, such as in the titering of antigen or antibody samples, for example in the selection of hybridomas.

In another example, the kits may be used to identify compounds that modulate expression of Neu5Gc-Lewis^{a} in in vitro disease cell models and/or in in vivo animal models, such as in, e.g., pancreatic or breast cancer cells or in vivo animal models for, e.g., pancreatic or breast cancer.

The following examples are included to demonstrate preferred embodiments of the disclosure. It should be appreciated by those of skill in the art that the techniques disclosed in the examples which follow represent techniques discovered by the inventor to function well in the practice of the disclosure, and thus may be considered to constitute preferred modes for its practice. However, those of skill in the art should, in light of the present disclosure, appreciate that many changes may be made in the specific embodiments which are disclosed and still obtain a like or similar result without departing from the spirit and scope of the disclosure.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that may be used to optimize the product purity and yield obtained from the described process. Only reasonable and routine experimentation will be required to optimize such process conditions.

### XIV. EXAMPLES

### Example 1

### Identification of Neu5Gc-Lewis^{a} as the Target for MGS5

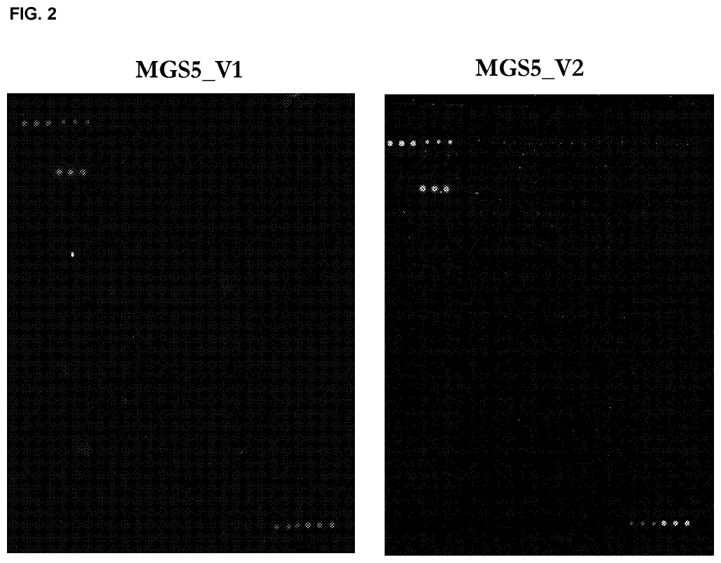

The Glycan Array 300 was obtained from (RayBiotech Life, GA-Glycan-300). The glycan array glass slide assembly was allowed to equilibrate to room temperature for 30 minutes. The slide was then removed from the package along with the cover film and allowed to air dry at room temperature for another 2 hours. The array was blocked for 30 minutes using the sample diluent provided in the kit. MGS5_V2 was diluted using the sample diluent to a concentration of 1 μg/ml and added to the array. The array was incubated for 3 hours at room temperature with gentle rocking. Following the 3-hour incubation the array was washed a total of 7 times with the provided wash buffers. The kit provided Cy3-Conjugated Streptavidin was added to the array for 1 hour at room temperature with gentle rocking. After the incubation the array was washed 5 times with the kit provided wash buffer. The array was disassembled and washed for 15 minutes with wash buffer, decanted, and then washed an additional five minutes. The array was washed with de-ionized water for 5 minutes. The array was then imaged using the Leica DMi8 microscope. Result: MGS5_V1 and MGS5_V2 are shown to strongly bind Neu5Gc-α-2,3-Gal-β-1,3-(Fuc-α-1,4)-GlcNAc (spotted in triplicate). FIG. 2.

### Example 2

### Identification of Neu5Gc-Lewis^{a} as a Biomarker for CANCER

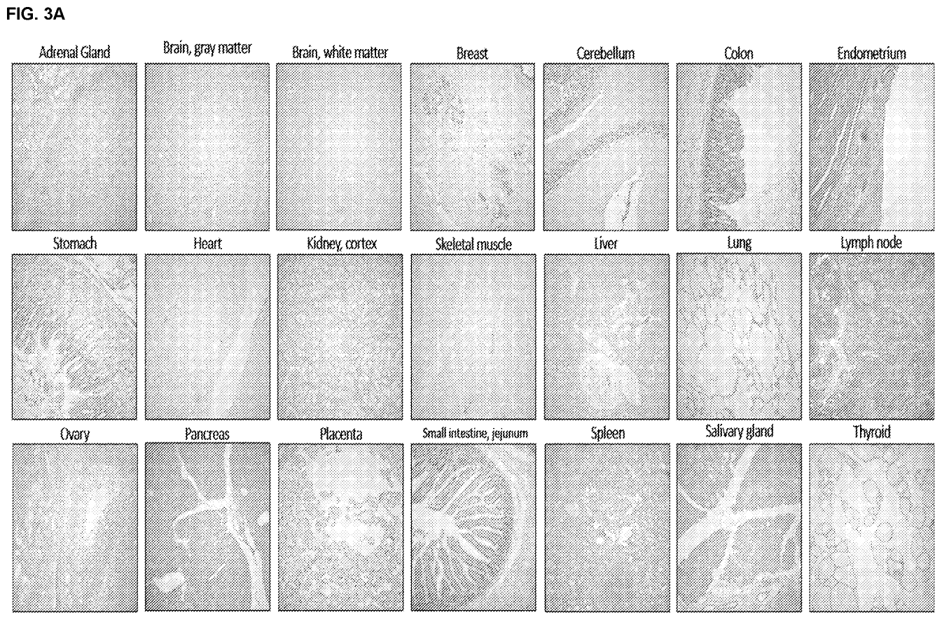

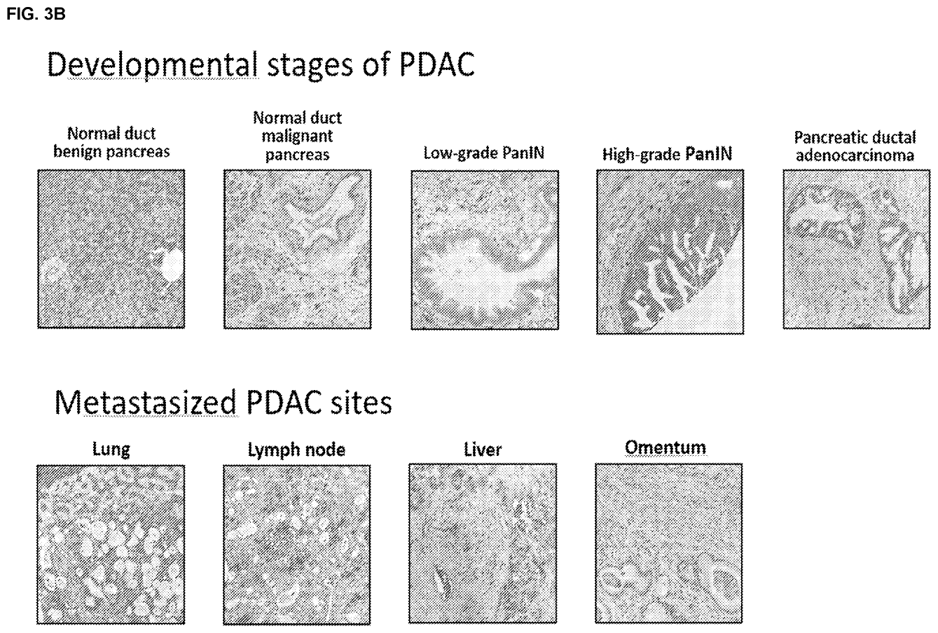

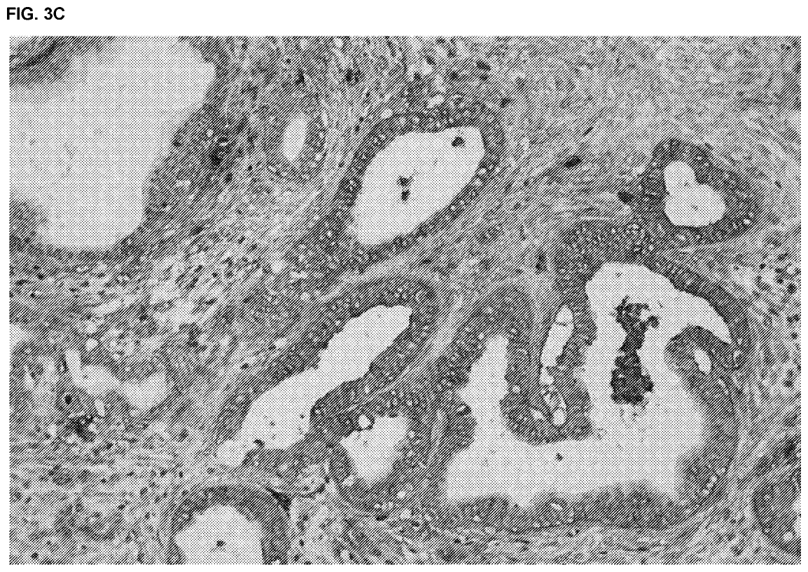

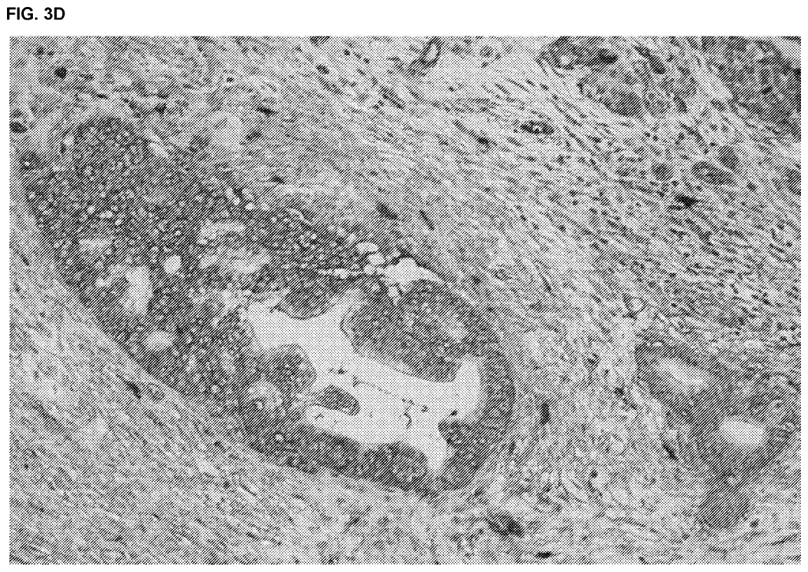

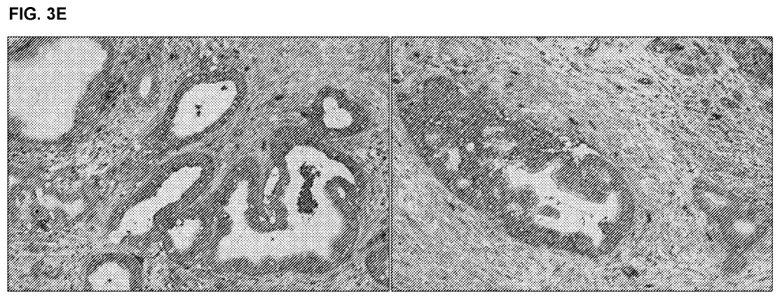

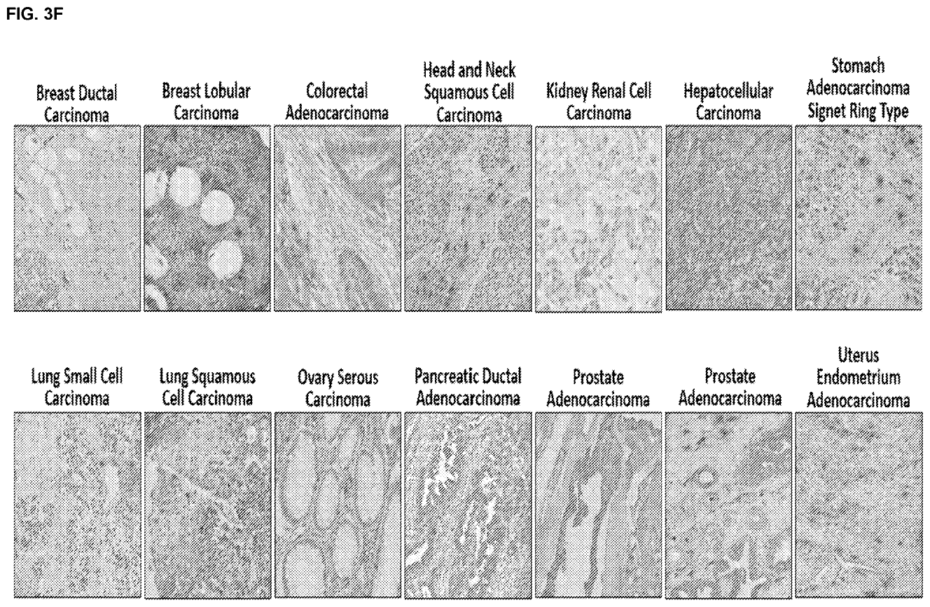

Tissue Microarrays were obtained from Novus Biologics and the Cooperative Human Tissue Network (CHTN). Slides were dried for 1 hour at 60° C. Tissues were deparaffinized by immersing in EZ-DeWax solution for 5 minutes, two times, with occasional agitation, and rinsed with water. Antigen retrieval was performed using citrate buffer pH 6.0 at 65° C. for 10 minutes. Slides were washed three times for 5 minutes each with 1×TBS. BLOXALL® (Vector Laboratories, SP-6000-100) was applied to the tissues for 10 minutes to block any endogenous peroxidases, followed by a 5-minute wash using 1×TBS. Slides were blocked for 30 minutes, followed by the addition of 25 nM MGS5-V2-HRP for 1 hour. Slides were washed 5 times for 5 minutes each using 1×TBS+0.1% Tween 20. To detect HRP signal, ImmPACT DAB solution (Vector Laboratories, SK-4105) was added to the tissues for 1 minute. Slides were rinsed in 0.05 M sodium bicarbonate, pH 9.6 for 10 minutes. Tissues were incubated with DAB enhancing solution (Vector Laboratories, H-2200) for 15 seconds, followed by a 5-minute wash in water. Slides were dipped in Hematoxylin (Gill's Formula) (Vector Laboratories, H-3401) for 3 minutes and rinsed in running tap water. Slides were differentiated by dipping 10 times in an acid rinse followed by 10 dips in tap water. Slides were incubated in a bluing solution for 1 minute and washed for 5 minutes in water. Rapid dehydration was performed using 2-propanol/isopropyl alcohol, twice, for 1 minute each. Coverslips were mounted using VectaMount® Express Mounting Medium (Vector Laboratories, H-5700-60) and allowed to dry. Slides were viewed and imaged using the Leica DMi8 microscope. FIG. 3.

### Example 3

Treatment of Cancer Cells with the Enzyme Sialidase Removes the Terminal Sialic Acid Residue from Glycans Present on its Surface

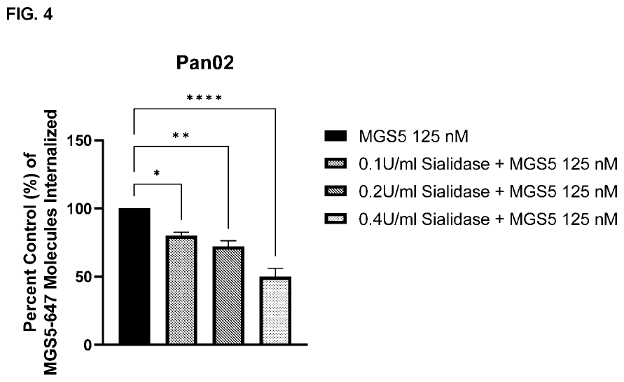

Pan02 cells were plated at 75,000 cells/well in a 12-well plate and allowed to attach for 24 hours. Following attachment, cells were treated with various concentrations of sialidase (Vibro cholerae) (Millipore Sigma, 11080725001) for 1 hour at 37° C. and pH 6.0. Sialidase treatment was removed and replaced with 125 nM of MGS5_V2-647 and incubated for 1 hour at 37° C. Following the 1 hour incubation, cells were washed with 1×PBS 3 times and prepared for flow cytometry. Result: Following sialidase treatment, MGS5 V2 showed up to a 50% reduction in cellular uptake by the murine pancreatic cancer cell line, Pan02. This result demonstrates MGS5_V2 dependence on Neu5Gc-Lewis^{a} for cellular internalization. FIG. 4.

### Example 4

### Assessment of the Binding Profile of MGS5_V2_2 Using a Human Plasma Membrane Protein Cell Array

The following describes the detailed procedure for the Retrogenix assay, which was used to determine MGS5's ability to bind to a variety of protein receptors.

### Pre-Screen

0.2 or 1 μg/mL of test peptide or PBS only was added to slides of fixed untransfected HEK293 cells using either the sequential method (test peptide added to slides, washed and then addition of AF647 Streptavidin detection reagent) or pre-incubation method (test peptide pre-incubated with AF647 Streptavidin at a 4:1 molar ratio before addition to slides) of secondary addition. Binding to untransfected cells was assessed by fluorescence imaging.

### Library Screen

For Library screening, 6052 expression vectors, encoding both ZsGreen1 and a full-length human plasma membrane protein, secreted or a cell surface-tethered human secreted protein, plus a further 397 human heterodimers were individually arrayed in duplicate across cell microarray slides (‘slide-sets’). An expression vector (pIRES-hEGFR-IRES-ZsGreen1) was spotted in quadruplicate on every slide and was used to ensure that a minimal threshold of transfection efficiency had been achieved or exceeded on every slide. Human HEK293 cells were used for reverse transfection/expression. Test peptide MGS5_V2 was added to each slide after cell fixation, using both the sequential and pre-incubation methods of secondary addition, giving a final concentration of 0.2 mg/mL. Detection of binding was performed by using AF647 Streptavidin. Fluorescent images were analyzed and quantitated (for transfection) using ImageQuant software (GE healthcare, Version 8.2). A protein ‘hit’ was defined as a duplicate spot showing a raised signal compared to background levels. This is achieved by visual inspection using the images gridded on the ImageQuant software. Hits were then classified as ‘strong, medium, weak or very weak’, depending on the intensity of the duplicate spots.

### Confirmation Screen

To confirm the hits and assess specificity, vectors encoding all hits identified in the Library screen, plus vectors encoding CD86 and EGFR, were arrayed and expressed in HEK293 cells on new slides. Confirmation screens and analyses were carried out as for Library screening except that identical slides were treated, either after cell fixation or in the absence of cell fixation, using both sequential and pre-incubation methods of secondary addition, with 0.2 μg/mL MGS5_V2, 1 μg/mL Biotinylated rhCTLA4 (positive control) or no test molecule (secondary only; negative control) (n=2 slides per treatment for fixed cell screen and n=1 slide per treatment for live cell screen). Binding to target-expressing cells and untransfected cells was assessed by fluorescence imaging.

### Results:

After screening 0.2 mg/mL of the test peptide for binding against fixed HEK293 cells expressing 6052 individual full-length human plasma membrane proteins, secreted and cell surface-tethered human secreted proteins, as well as a further 397 human heterodimers using both the sequential and pre-incubation methods of secondary addition, 16 library hits were identified altogether. Twelve of the 16 library hits were bound by MGS5_V2 and at least 1 of the control treatments (Biotinylated rhCTLA4 and/or no primary test molecule). These are therefore classed as non-specific. A further 2 of the 16 library hits appeared to be specific to MGS5 V2. However, signal intensities were classed as very weak and, in each instance, only observed on 1 of the 2 replicate slides. For such hits, signals are so close to background levels that there is little or no confidence that they are real and/or specific. No significance should be attributed to such hits. After excluding the non-reproducible, non-specific and non-significant hits, no interactions remained that were specific to the test peptide using the sequential method of secondary addition. In a Confirmation screen performed on live cells in the absence of cell fixation, MGS5_V2 showed no specific interactions using either the sequential or pre-incubation methods of secondary addition.

### Conclusions

Following a screen for binding against fixed HEK293 cells expressing 6052 individual full-length human plasma membrane proteins, secreted and cell surface-tethered human secreted proteins, as well as a further 397 human heterodimers, followed by a series of confirmatory screens, MGS5_V2 showed no specific interactions using either the sequential or pre-incubation methods of secondary addition on either fixed or live cell microarrays.

### Example 5

### Screening of Other Diseases for the Presence of Neu5Gc-Lewis^{a}

The disclosure provides methods for diagnosing and treating cancer and provides evidence that Neu5Gc-Lewis^{a} is present in cancer cells and tissues but not in their healthy counterparts. However, the methods may be used to treat other diseases or conditions, provided that they are first identified as associated with the presence of Neu5Gc-Lewis^{a}. Although it may be preferable that Neu5Gc-Lewis^{a} presence be limited to cancer cells and tissues for purposes of drug targeting and diagnosis, the methods of the disclosure provide that the presence of Neu5Gc-Lewis^{a} may be associated with, although it may be confirmed, a pathological cell in an organ or tissue selected from the group consisting of liver, lung, kidney, brain, intestine, spleen, heart, muscle, and lymph node. In some embodiments, the disease or condition is selected from the group consisting of diabetes, autoimmune diseases, hematological diseases, cardiac diseases, vascular diseases, inflammatory diseases, fibrotic diseases, viral infectious diseases, hereditary diseases, ocular diseases, liver diseases, lung diseases, muscle diseases, protein deficiency diseases, lysosomal storage diseases, neurological diseases, kidney diseases, aging and degenerative diseases, and diseases characterized by cholesterol level abnormality.

### Example 6

### Conjugation of MGS5

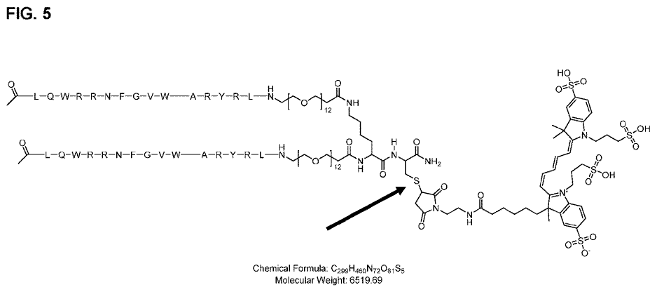

The disclosure provides conjugates of MGS5. FIG. 5 shows an example of a conjugate formed by reaction of the thiol group (highlighted by arrow) on the dimer core with the cargo, in this example AF647.

### Example 7

### Quantitation of Neu5Gc-Lewis^{a} on Exosomes

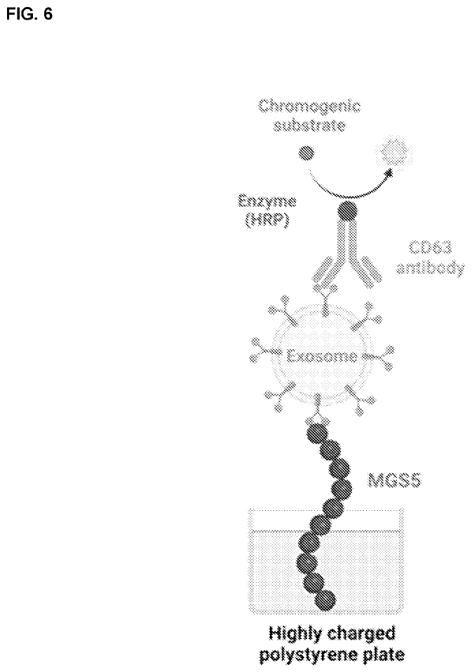

The disclosure provides compositions and methods of quantifying Neu5Gc-Lewis^{a} on exosomes. FIG. 6 shows an example of an assay to detect Neu5Gc-Lewis^{a} on exosomes. An exemplary protocol may include the steps of:
contacting a surface bound with an Neu5Gc-Lewis^{a} binding molecule with a sample comprising an exosome (e.g., an ELISA plate coated with MGS5 peptide), to capture exosomes comprising Neu5Gc-Lewis^{a};
contacting the captured exosomes with anti-exosome antibody conjugated to a molecule for detection of the captured exosomes (e.g., an anti-CD63 antibody linked to a HRP enzyme); and
detecting the binding of the secondary antibody to the captured exosomes (e.g., developed using HRP chromogenic substrate.)

For example, when HRP is used for detection, color development is dependent upon level of MGS5 capture of Neu5Gc-Lewis^{a} on exosomes present in the sample (e.g., blood-borne exosomes released by tumor cells).

While various specific embodiments/embodiments have been illustrated and described, it will be appreciated that various changes may be made without departing from the spirit and scope of the disclosure.

All publications, patents, patent applications and other documents cited in this application are hereby incorporated by reference in their entireties for all purposes to the same extent as if each individual publication, patent, patent application or other document were individually indicated to be incorporated by reference for all purposes.

1. A composition comprising at least one Neu5Gc-Lewis^{a}-binding molecule, wherein the Neu5Gc-Lewis^{a}-binding molecule does not bind to any other form of Neu5Gc or any other Sialyl-Lewis^{a}, including free Neu5Gc.

2. The composition of claim 1, comprising a peptide, antibody, nucleic acid, a carbohydrate, a lipid, a small organic molecule, a lipid particle, a nanoparticle, or an aptamer.

3. The composition of claim 1, comprising a peptide having at least 75%, 80%, 85%, 90%, 95%, or 100% identity to a peptide comprising the sequence of SEQ ID NO:1 or SEQ ID NO:2.

4. The composition of any one of claims 1 to 3, wherein the Neu5Gc-Lewis^{a}-binding molecule is chemically modified.

5. The composition of claim 4, wherein the chemical modification comprises pegylation, acetylation, a d-amino acid, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphatidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, proteolytic processing, phosphorylation, prenylation, racemization, selenoylation, sulfation, arginylation, or transfer-RNA mediated addition of amino acids to protein, or any combination thereof.

6. The composition of any one of claims 1 to 5 comprising at least two linked Neu5Gc-Lewis^{a}-binding molecules.

7. The composition of claim 6, wherein the at least two Neu5Gc-Lewis^{a}-binding molecules are linked to each other through a linker.

8. The composition of claim 7, wherein the linker comprises PEG.

9. A method of detecting Neu5Gc-Lewis^{a} in a sample, comprising contacting the sample with a composition of any one of claims 1 to 8, and detecting binding between Neu5Gc-Lewis^{a} and the Neu5Gc-Lewis^{a}-binding molecule.

10. The method of claim 9, wherein the method comprises an in vitro assay.

11. The method of claim 9, wherein the sample comprises a cell, exosome or tissue from a subject having or at risk of cancer.

12. The method of claim 9, wherein the sample comprises tissue sample, and further wherein the method comprises detecting binding between Neu5Gc-Lewis^{a} and the Neu5Gc-Lewis^{a}-binding molecule in the extracellular matrix of the tissue sample.

13. A method of diagnosing cancer in a subject, the method comprising contacting a cell or tissue from the subject with a composition of any one of claims 1 to 8, and detecting the presence of Neu5Gc-Lewis^{a} in the cell or tissue sample from the subject, wherein the presence of Neu5Gc-Lewis^{a} in the cell or tissue indicates the presence of a cancer cell or tissue, and diagnosing cancer in the subject.

14. The method of claim 13, further comprising administering an anti-cancer therapy to the subject.

15. The method of any one of claims 13 to 14, wherein the cancer is pancreatic cancer, breast cancer, colon cancer, brain cancer, endometrial cancer, liver cancer, lung cancer, ovarian cancer, prostate cancer, stomach cancer, or urinary bladder cancer.

16. The method of any one of claims 13 to 15, wherein the presence of Neu5Gc-Lewis^{a} discriminates between normal and cancerous tissue.

17. A method of targeted delivery of a cargo molecule to a cell and/or tissue, comprising contacting the cell or tissue with a composition of any one of claims 1-8 comprising a Neu5Gc-Lewis^{a}-binding molecule linked to a cargo, directly or through a linker.

18. The method of claim 17, wherein the cell or tissue is a cancer cell or tissue.

19. The method of claim 17 or 18, wherein the cancer is pancreatic cancer, breast cancer, colon cancer, brain cancer, endometrial cancer, liver cancer, lung cancer, ovarian cancer, prostate cancer, stomach cancer, or urinary bladder cancer.

20. The method of any one of claims 17 to 19, wherein the cargo is an anti-cancer therapeutic agent.

21. The method of claim 20, wherein the cargo comprises a molecule selected from a peptide, a nucleic acid molecule, an siRNA, an antisense oligonucleotide, a microRNA, an shRNA, a protein, a blood clotting factor, an anticoagulant, a bone morphogenetic protein, an engineered protein scaffold, a thrombolytic protein, a CRISPR protein, granulocyte-macrophage colony-stimulating factor (GMCSF), a transcription factor, a transposon, a reverse transcriptase, a viral interferon antagonist, a fluorophore, a liposome, polysaccharide, a nanoparticle, a small molecule therapeutic, a PET dye, an NIR dye, an MRI agent, an antibody, a ribonucleoprotein, an antigenic peptide, a protein toxin, or a genome editing system.
